(19) United States
(12) Patent Application Publication
(10) Pub. No.: US 2016/0008277 A1
(43) Pub. Date: Jan. 14, 2016

(54) CO-PACKAGED DRUG PRODUCTS

(71) Applicant: Lightlake Therapeutics Inc., New York, NY (US)

(72) Inventors: Roger Crystal, Santa Monica, CA (US); Michael Brenner Weiss, Manhattan, NY (US)

(21) Appl. No.: 14/795,403

(22) Filed: Jul. 9, 2015

Related U.S. Application Data
(60) Provisional application No. 62/022,268, filed on Jul. 9, 2014.

(51) Int. Cl. A61K 9/00; A61K 47/02; A61M 15/00; A61K 45/06; A61M 15/08; A61K 31/485; A61K 47/18

(52) U.S. Cl. CPC A61K 9/0043; A61K 31/485; A61K 47/02; A61K 47/186; A61K 47/183; A61K 45/06; A61M 15/08; A61M 15/009; A61M 2205/58

(57) ABSTRACT

Provided herein are drug products comprising a combination of an opioid receptor agonist and an opioid receptor agonists, including co-packaged drug products comprising an opioid receptor agonist and an opioid receptor antagonist in a delivery device selected from a hand-held auto-injector and a pre-primed nasal delivery device, and methods of prescribing and dispensing same. Methods of lowering opioid overdose risk by providing a combination of an opioid receptor agonist and an opioid receptor agonists, for example by co-packaging an opioid receptor agonist and an opioid receptor antagonist in a delivery device, are also provided.

This application claims the benefit of U.S. Provisional Application No. 62/022,268, filed Jul. 9, 2014, the disclosure of which is hereby incorporated by reference as if written herein in its entirety.

Provided are co-packaged drug products comprising an opioid receptor agonist, and an opioid receptor antagonist in a delivery device selected from a hand-held auto-injector and a pre-primed nasal delivery device, and methods of prescribing and dispensing same. Also provided are methods of lowering opioid overdose risk by co-packaging an opioid receptor agonist with an opioid receptor antagonist delivery device.

Opioid receptors are G protein-coupled receptors (GPCRs) that are activated both by endogenous opioid peptides and by clinically important alkaloid analgesic drugs such as morphine. There are three principal types of opioid receptors: the δ-opioid receptor, the κ-opioid receptor, and the μ-opioid receptor. Opioids (i.e. opioid agonists or opioid receptor agonists) depress respiration, which is controlled principally through medullary respiratory centers with peripheral input from chemoreceptors and other sources. Opioids produce inhibition at the chemoreceptors via μ-opioid receptors and in the medulla via μ- and δ-opioid receptors. While there are a number of neurotransmitters mediating the control of respiration, glutamate and γ-aminobutyric acid (GABA) are the major excitatory and inhibitory neurotransmitters, respectively. This explains the potential for interaction of opioids with benzodiazepines and alcohol: both benzodiazepines and alcohol facilitate the inhibitory effect of GABA at the GABA_{A} receptor, while alcohol also decreases the excitatory effect of glutamate at NMDA receptors. Oxycodone and other opioid painkillers, as well as heroin and methadone are all implicated in fatal overdose. Heroin has three metabolites with opioid activity. Variation in the formation of these metabolites due to genetic factors and the use of other drugs could explain differential sensitivity to overdose. Metabolites of methadone contribute little to its action. However, variation in rate of metabolism due to genetic factors and other drugs used can modify methadone concentration and hence overdose risk. The degree of tolerance also determines risk. Tolerance to respiratory depression is less than complete, and may be slower than tolerance to euphoric and other effects. One consequence of this may be a relatively high risk of overdose among experienced opioid users. While agonist administration modifies receptor function, changes (usually in the opposite direction) also result from use of antagonists, for example, supersensitivity to opioids following a period of administration of antagonists such as naltrexone.

In the United States, mortality rates closely correlate with opioid sales. In 2008, approximately 36,450 people died from drug overdoses. At least 14,800 of these deaths involved prescription opioid analgesics. Moreover, according to the Substance Abuse and Mental Health Services Administration, the number/rate of Americans 12 years of age and older who currently abuse pain relievers has increased by 20 percent between 2002 and 2009. In New York City, between 1990 and 2006, the fatality rate from prescription opioids increased seven-fold, from 0.39 per 100,000 persons to 2.7. Drugs classed as prescription opioids in this study include both typical analgesics, such as OxyContin® (oxycodone HCl controlled-release) and methadone (used in the treatment of dependence on other opioids such as heroin and also prescribed for pain), but the increase in the rate of drug overdose over the 16 years of the study was driven entirely by overdoses of typical analgesics. Over the same time period, methadone overdoses remained stable, and overdoses from heroin declined. Whites were more likely than blacks and Latinos to overdose on these analgesics, and deaths mostly occurred in neighborhoods with lower rates of poverty, suggesting differential access to doctors who can write painkiller prescriptions may be a driving force behind the racial disparity. (Cerdá et al. “Prescription opioid mortality trends in New York City, 1990-2006: Examining the emergence of an epidemic,” Drug and Alcohol Dependence Volume 132, Issues 1-2, 1 Sep. 2013, 53-62.)

Naloxone is an opioid receptor antagonist that is approved for use by injection for the reversal of opioid overdose and for adjunct use in the treatment of septic shock. It is currently being used mainly in emergency departments and in ambulances by trained medical professionals. There have been efforts to expand its use by providing the drug to some patients with take-home opioid prescriptions and those who inject illicit drugs, potentially facilitating earlier administration of the drug. The UN Commission on Narcotics Drugs “encourages all Member States to include effective elements for the prevention and treatment of drug overdose, in particular opioid overdose, in national drug policies, where appropriate, and to share best practices and information on the prevention and treatment of drug overdose, in particular opioid overdose, including the use of opioid receptor antagonists such as naloxone.”

U.S. Pat. No. 4,464,378 describes a method for eliciting an analgesic or narcotic antagonist response in a warm-blooded animal, which comprises administering intranasally (IN) to said animal to elicit a narcotic antagonist response, a narcotic antagonist effective amount of naloxone. WO 82/03768 discloses a composition that contains 1 mg of naloxone hydrochloride per 0.1 ml of solution adapted for nasal administration used in the treatment of narcotic induced respiratory depression (overdose) at a dosage approximately the same as that employed for intravenous (IV), intramuscular (IM) or subcutaneous (SQ) administration. WO 00/62757 teaches pharmaceutical compositions for IN or oral (PO) administration which comprise an opioid antagonist, such as naloxone for application by spray in the reversal of opioid depression for treatment of patients suffering from opioid over-dosage, wherein the spray applicator is capable of delivering single or multiple doses and suitable dosage units are in the range of 0.2 to 5 mg.

The use of nasal naloxone is not without controversy. For instance, Loimer et al. (International Journal of Addictions, 29(6), 819-827, 1994) reported that the nasal administration of naloxone is as effective as the intravenous route in opiate addicts, however, Dowling et al. (Ther Drug Monit, Vol 30, No 4, August 2008) reported that naloxone administered intranasally displays a relative bioavailability of 4% only and concluded that the IN absorption is rapid but does not maintain measurable concentrations for more than an hour.

One early study of 196 consecutive patients with suspected opioid overdose conducted in an urban out-of-hospital setting, had shown the mean interval from emergency medical services (EMS) arrival to a respiratory rate of ≧10 breaths/min was 9.3±4.2 min with administration of naloxone 0.4 mg IV, versus 9.6±4.58 min with administration of naloxone 0.8 mg SQ. The authors concluded that the slower rate of absorption via the SQ route was offset by the delay in establishing an IV line. (Wanger et al., Intravenous vs subcutaneous naloxone for out-of-hospital management of presumed opioid overdose. Acad Emerg Med. 1998 April; 5(4):293-9).

The Denver Health Paramedic system subsequently investigated the efficacy and safety of atomized intranasal naloxone for the treatment of suspected opiate overdose (Barton, et al., Efficacy of intranasal naloxone as a needleless alternative for treatment of opioid overdose in the prehospital setting. J Emerg Med, 2005. 29(3): p. 265-71). All adult patients encountered in the prehospital setting as suspected opiate overdose, found down, or with altered mental status who met the criteria for naloxone administration were included in the study. IN naloxone (2 mg) was administered immediately upon patient contact and before IV insertion and administration of IV naloxone (2 mg). Patients were then treated by EMS protocol. The main outcome measures were: time of IN naloxone administration, time of IV naloxone administration, time of appropriate patient response as reported by paramedics. Ninety-five patients received IN naloxone and were included in the study. A total of 52 patients responded to naloxone by either IN or IV, with 43 (83%) responding to IN naloxone alone. Seven patients (16%) in this group required further doses of IV naloxone. The median times from arrival at patient side to awakening and from administration of the IN naloxone to patient awakening were 8.0 minutes and 3.0 minutes respectively.

The Drug Overdose Prevention and Education (DOPE) Project was the first naloxone prescription program (NPP) established in partnership with a county health department (San Francisco Department of Public Health), and is one of the longest running NPPs in the USA. From September 2003 to December 2009, 1,942 individuals were trained and prescribed naloxone through the DOPE Project, of whom 24% returned to receive a naloxone refill, and 11% reported using naloxone during an overdose event. Of 399 overdose events where naloxone was used, participants reported that 89% were reversed. In addition, 83% of participants who reported overdose reversal attributed the reversal to their administration of naloxone, and fewer than 1% reported serious adverse effects. Findings from the DOPE Project add to a growing body of research that suggests that intravenous drug users (IDUs) at high risk of witnessing overdose events are willing to be trained on overdose response strategies and use take-home naloxone during overdose events to prevent deaths (Enteen, et al., Overdose prevention and naloxone prescription for opioid users in San Francisco. J Urban Health. 2010 December; 87(6):931-41).

Another reported study reviewed EMS and hospital records before and after implementation of a protocol for administration of intranasal naloxone by the Central California EMS Agency in order to compare the prehospital time intervals from patient contact and medication administration to clinical response for IN versus intravenous IV naloxone in patients with suspected narcotic overdose. The protocol for the treatment of opioid overdose with intranasal naloxone was as follows: “Intranasal (IN)—Administer 2 mg intranasally (1 mg per nostril) using mucosal atomizer device (MAD™) if suspected narcotic intoxication and respiratory depression (rate 8 or less). This dose may be repeated in 5 minutes if respiratory depression persists. Respirations should be supported with a bag valve mask until respiratory rate is greater than 8. Intramuscular (IM)—Administer 1 mg if unable to administer intranasally (see special considerations). May repeat once in 5 minutes. Intravenous (IV)—Administer 1 mg slow IV push if no response to intranasal or IM administration after 10 minutes. Pediatric dose—0.1 mg/kg intranasally, if less than 10 kg and less than 1 year old”. Patients with suspected narcotic overdose treated in the prehospital setting over 17 months, between March 2003 and July 2004 were included. Paramedics documented dose, route of administration, and positive response times using an electronic record. Clinical response was defined as an increase in respiratory rate (breaths/min) or Glasgow Coma Scale score of at least 6. Main outcome variables included time from medication to clinical response and time from patient contact to clinical response. Secondary variables included numbers of doses administered and rescue doses given by an alternate route. Between-group comparisons were accomplished using t-tests and chi-square tests as appropriate. One hundred fifty-four patients met the inclusion criteria, including 104 treated with IV and 50 treated with IN naloxone. Clinical response was noted in 33 (66%) and 58 (56%) of the IN and IV groups, respectively (p=0.3). The mean time between naloxone administration and clinical response was longer for the IN group (12.9 vs. 8.1 min, p=0.02). However, the mean times from patient contact to clinical response were not significantly different between the IN and IV groups (20.3 vs. 20.7 min, p=0.9). More patients in the IN group received two doses of naloxone (34% vs. 18%, p=0.05), and three patients in the IN group received a subsequent dose of IV or IM naloxone. (Robertson et al., Intranasal naloxone is a viable alternative to intravenous naloxone for prehospital narcotic overdose. Prehosp Emerg Care. 2009 October-December; 13(4):512-5).

In August 2006, the Boston Public Health Commission passed a public health regulation that authorized an opioid overdose prevention program that included intranasal naloxone education and distribution of the spray to potential bystanders. Participants were instructed by trained staff to deliver 1 mL (1 mg) to each nostril of the overdose victim. After 15 months, the program had provided training and intranasal naloxone to 385 participants who reported 74 successful overdose reversals (Doe-Simkins et al. Overdose prevention education with distribution of intranasal naloxone is a feasible public health intervention to address opioid overdose. Am J Public Health. 2009; 99:788-791).

Overdose education and nasal naloxone distribution (OEND) programs are community-based interventions that educate people at risk for overdose and potential bystanders on how to prevent, recognize and respond to an overdose. They also equip these individuals with a naloxone rescue kit. To evaluate the impact of OEND programs on rates of opioid related death from overdose and acute care utilization in Massachusetts, an interrupted time series analysis of opioid related overdose death and acute care utilization rates from 2002 to 2009 was performed comparing community-year strata with high and low rates of OEND implementation to those with no implementation. The setting was nineteen Massachusetts communities (geographically distinct cities and towns) with at least five fatal opioid overdoses in each of the years 2004 to 2006. OEND was implemented among opioid users at risk for overdose, social service agency staff, family, and friends of opioid users. OEND programs equipped people at risk for overdose and bystanders with nasal naloxone rescue kits and trained them how to prevent, recognize, and respond to an overdose by engaging emergency medical services, providing rescue breathing, and delivering naloxone. Among these communities, OEND programs trained 2,912 potential bystanders who reported 327 rescues. Both community-year strata with 1-100 enrollments per 100,000 population (adjusted rate ratio 0.73, 95% confidence interval 0.57 to 0.91) and community-year strata with greater than 100 enrollments per 100,000 population (0.54, 0.39 to 0.76) had significantly reduced adjusted rate ratios compared with communities with no implementation. Differences in rates of acute care hospital utilization were not significant. Opioid overdose death rates were reduced in communities where OEND was implemented. This study provides observational evidence that by training potential bystanders to prevent, recognize, and respond to opioid overdoses, OEND is an effective intervention (Walley et al., Opioid overdose rates and implementation of overdose education and nasal naloxone distribution in Massachusetts: interrupted time series analysis. BMJ 2013; 346:f174).

Naloxone prescription programs are also offered by community-based organizations in Los Angeles and Philadelphia. Programs in both cities target IDUs. Studies which recruited 150 IDUs across both sites for in-depth qualitative interviews compared two groups of IDUs, those who had received naloxone prescriptions and those who had never received naloxone prescriptions. In both L.A. and Philadelphia, IDUs reported successfully administering naloxone to reverse recently witnessed overdoses. Reversals often occurred in public places by both housed and homeless IDUs. Despite these successes, IDUs frequently did not have naloxone with them when they witnessed an overdose. Two typical reasons reported were naloxone was confiscated by police, and IDUs did not feel comfortable carrying naloxone in the event of being stopped by police. Similarly, some untrained IDUs reported discomfort with the idea of carrying naloxone on them as their reason for not gaining a prescription.

A randomized trial comparing 2 mg naloxone delivered intranasally with a mucosal atomizer to 2 mg intramuscular naloxone was reported by Kelly et al., in 2005 (Med J Aust. 2005 Jan. 3; 182(1):24-7). The study involved 155 patients (71 IM and 84 IN) requiring treatment for suspected opiate overdose and attended by paramedics of the Metropolitan Ambulance Service (MAS) and Rural Ambulance Victoria in Victoria, Australia. The IM group had more rapid response than the IN group, and were more likely to have more than 10 spontaneous respirations per minute within 8 minutes (82% v. 63%; P=0.0173). There was no statistically significant difference between the IM and IN groups for needing rescue naloxone (13% [IM group] v. 26% [IN group]; P=0.0558). The authors concluded that IN naloxone is effective in treating opiate-induced respiratory depression, but is not as effective as IM naloxone.

Kerr et al. (Addiction. 2009 December; 104(12):2067-74) disclosed treatment of heroin overdose by intranasal administration of naloxone constituted in a vial as a preparation of 2 mg in 1 mL. Participants received 1 mg (0.5 ml) in each nostril. The rate of response within 10 minutes was 60/83 (72.3%) for 2 mg IN naloxone versus 69/89 (77.5%) for 2 mg IM naloxone. The mean response times were 8.0 minutes and 7.9 minutes for IN and IV naloxone respectively. Supplementary naloxone was administered to fewer patients who received IM naloxone (4.5%) than IN (18.1%).

WO2012156317 describes a study in which naloxone, 8 mg and 16 mg, was administered as 400 μL IN (200 μL per nostril). The administration was performed as follows: The pump of the nasal spray was primed by removing the cap and pressing downward. This is repeated at least 6 times or until a fine spray appears; priming is done just prior to dosing. The subject is in a standing or upright position and should gently blow the nose to clear the nostrils. The subject should tilt the head forward slightly and gently close one nostril by pressing the outside of the nose with a finger on the nostril to be closed. The device is inserted into the open nostril and it is sprayed 2 times into the nostril. The subject should gently breath inward through the nostril, the device is removed, and the steps are repeated for the other nostril. The mean Tₘₐₓ values were reported to be 0.34 h (20.4 min) and 0.39 h (23.4 min) for the 8 and 16 mg doses respectively.

Wermeling (Drug Deliv Transl Res. 2013 Feb. 1; 3(1): 63-74) teaches that the initial adult dose of naloxone in known or suspected narcotic overdose is 0.4 to 2 mg, which may be repeated to a total dose of 10 mg and that the current formulations of naloxone are approved for intravenous (IV), intramuscular (IM) and subcutaneous (SC) administration, with IV being the recommended route. Wermeling also predicts that a 2 mg nasal solution dose of naloxone will likely have a Cₘₐₓ of 3-5 ng/mL and a tₘₐₓ of approximately 20 minutes.

Since the onset of action of naloxone used in opioid overdose cases should be as fast as possible, naloxone is thus far mainly administered intravenously or intramuscularly by emergency health care personnel. Due to a high first pass metabolism, oral dosage forms comprising naloxone display a low bioavailability and thus seem to be not suitable for such purposes. The administration of naloxone via injection into the blood stream or into the muscle requires first of all trained medical personnel (for intravenous injection) or a trained carer (for intramuscular injection). Secondly, depending on the constitution of the addict and the period of intravenous drug abuse, it can be particularly difficult to find access into a vein of the addict's body for administering naloxone intravenously. Clearly, there is a risk of exposure to blood borne pathogens for the medical personnel or the trained carer since a large population of drug addicts suffers from blood borne pathogen induced diseases such as HIV, hepatitis B and C, and the like since accidental needlestick is a serious safety concern. 385,000 needle-stick injuries have been estimated to have occurred in the year 2000 in the US alone (Wilburn, Needlestick and sharps injury prevention, Online J Issues Nurs 2004, Sep. 30; 9(3):5).

Naloxone has a relatively short half-life of compared to some longer-acting opioid formulations and so after a typical therapeutic dose of naloxone is administered to an opioid overdose patient there is often the need to re-administer naloxone, in some cases even several times, and it is important to seek immediate medical attention.

Furthermore, it has been suggested that in view of the growing opioid overdose crisis in the US, naloxone should be made available over-the-counter (OTC), which would require a device, such as a nasal spray device, that untrained consumers are able to use safely. A nasal spray device that was pre-filled with a naloxone formulation would also be less likely to be confiscated by police than the system developed by some EMS programs that combines an U.S. Food and Drug Administration (FDA)-approved naloxone injection product with a marketed, medical device called the Mucosal Atomization Device.

On Apr. 2, 2014, the FDA approved a prescription treatment that can be used by family members or caregivers to treat a person known or suspected to have had an opioid overdose. Evzio™ (naloxone hydrochloride injection) rapidly delivers a single dose of the drug naloxone via a hand-held auto-injector that can be carried in a pocket or stored in a medicine cabinet.

Drug products adapted for nasal delivery comprising a pre-primed device and a pharmaceutical composition comprising an opioid receptor antagonist, and methods of use thereof are described in U.S. provisional application 61/953,379, which is herein incorporated by reference in its entirety.

There remains a need to lower the risk of opioid overdose. The co-packaged drug products and methods of prescribing and dispensing same described herein meet this and other needs.

Provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a drug delivery device selected from: a pre-primed device adapted for nasal delivery of a pharmaceutical composition to a patient, and a hand-held auto-injector adapted for intramuscular or subcutaneous delivery of a pharmaceutical composition to a patient.

Also provided are methods of lowering opioid overdose risk comprising the steps of: co-packaging a therapeutically effective amount of an opioid agonist, and a therapeutically effective amount of an opioid antagonist selected from: naloxone and pharmaceutically acceptable salts thereof, to form a co-packaged drug product; prescribing the co-packaged drug product to a first individual; and dispensing the co-packaged drug product to the first individual or a second individual; wherein the opioid antagonist is contained in a drug delivery device selected from: a pre-primed device adapted for nasal delivery of a pharmaceutical composition to a patient, and a hand-held auto-injector adapted for intramuscular or subcutaneous delivery of a pharmaceutical composition to a patient.

### BRIEF DESCRIPTION OF THE DRAWINGS

FIG. 1 shows the mean (±SD) naloxone plasma concentration following administration of 0.4 mg intramuscular (IM), 2 mg intranasal (IN), and 4 mg intranasal in 14 human subjects.

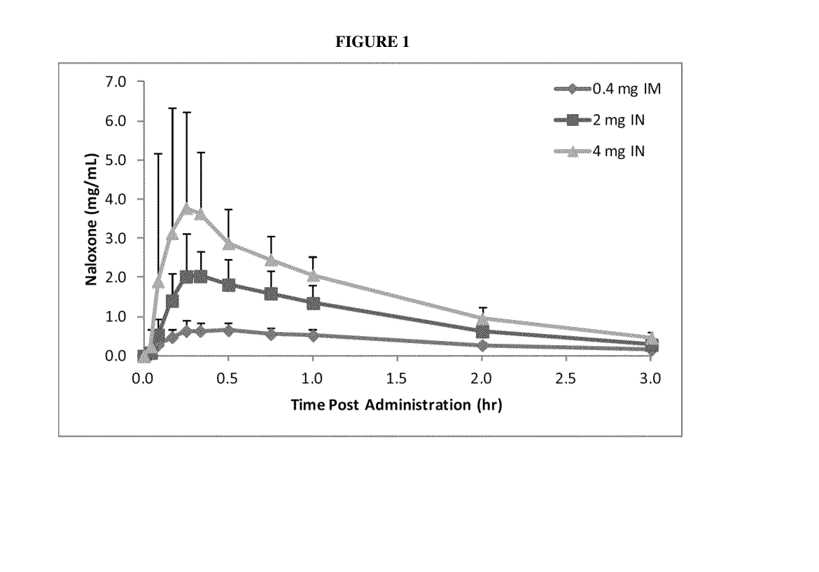

FIG. 2 shows the mean (±SD) naloxone plasma concentration with logarithmic transformation following administration of 0.4 mg intramuscular (IM), 2 mg intranasal (IN), and 4 mg intranasal in 14 human subjects.

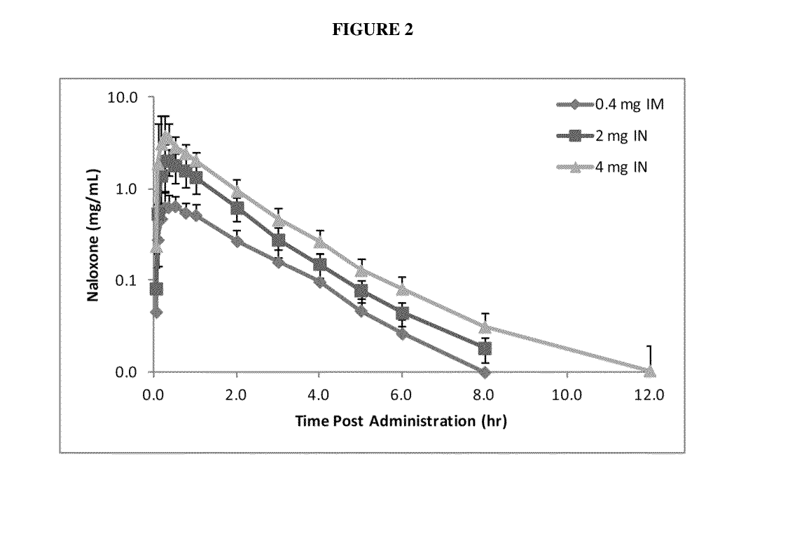

FIG. 3 shows the mean naloxone plasma concentration following single intranasal administrations (FIG. 3A) and intramuscular injections (FIG. 3B) of naloxone to healthy subjects (N=28) over a twelve-hour period.

FIG. 4 shows the mean naloxone plasma concentration following single intranasal administrations (FIG. 4A) and intramuscular injections (FIG. 4B) of naloxone to healthy subjects (N=28) over a four-hour period.

FIG. 5 shows the mean naloxone plasma concentration following intramuscular injection of 0.4 mg naloxone (FIG. 5A, top) and one spray of 20 mg/mL naloxone (FIG. 5B, bottom) to healthy male (N=16) and female (N=12) subjects over a twelve-hour period.

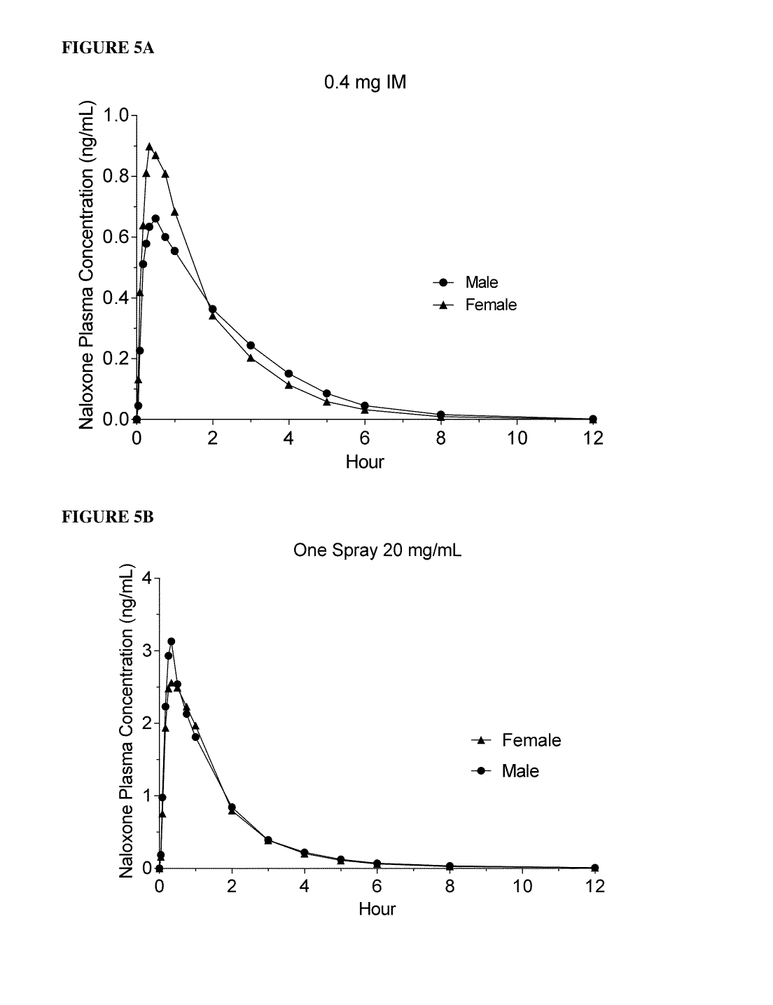

FIG. 6 shows the mean naloxone plasma concentration following two sprays of 20 mg/mL (FIG. 6A, top) and one spray of 40 mg/mL (FIG. 6B, bottom) to healthy male (N=16) and female (N=12) subjects over a twelve-hour period.

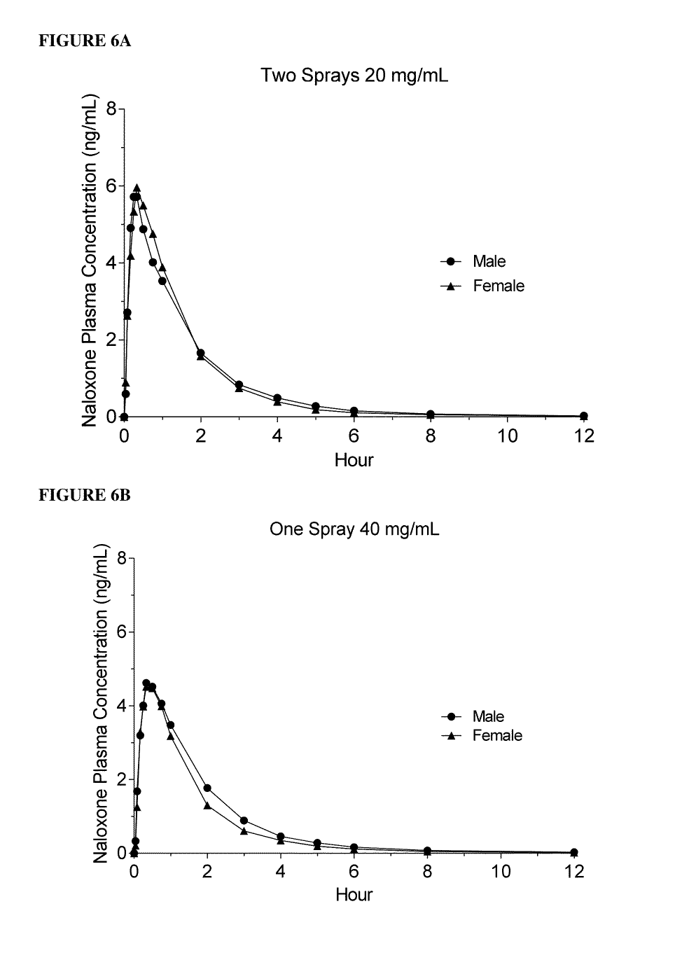

FIG. 7 shows the mean naloxone plasma concentration following two sprays of 40 mg/mL to healthy male (N=16) and female (N=12) subjects over a twelve-hour period.

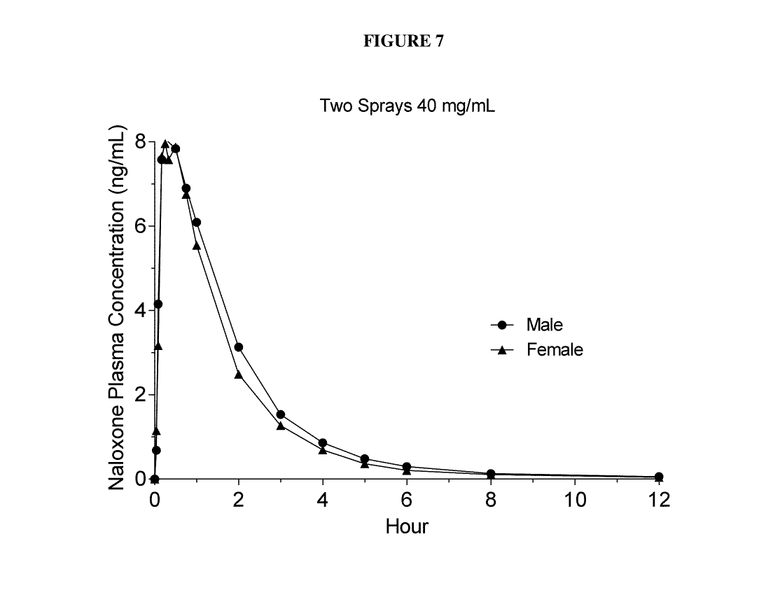

### DETAILED DESCRIPTION

Provided herein is a drug product comprising a combination of a therapeutically effective amount of an opioid agonist and a therapeutically effective amount of naloxone hydrochloride or a hydrate thereof, wherein said naloxone hydrochloride or hydrate thereof is contained in drug delivery device selected from a pre-primed device adapted for nasal delivery of a pharmaceutical composition to a patient, and a hand-held auto-injector adapted for intramuscular or subcutaneous delivery of a pharmaceutical composition to a patient.

In certain embodiments disclosed herein, including in paragraph [034], the combination of a therapeutically effective amount of an opioid agonist and a therapeutically effective amount of naloxone hydrochloride or a hydrate thereof, wherein said naloxone hydrochloride or hydrate thereof is contained in drug delivery device selected from a pre-primed device adapted for nasal delivery of a pharmaceutical composition to a patient and a hand-held auto-injector adapted for intramuscular or subcutaneous delivery of a pharmaceutical composition to a patient, is co-packaged.

Also provided herein is a co-packaged drug product comprising a therapeutically effective amount of an opioid agonist, and a therapeutically effective amount of an opioid antagonist selected from naloxone and pharmaceutically acceptable salts thereof; wherein said opioid antagonist is contained in a drug delivery device selected from a pre-primed device adapted for nasal delivery of a pharmaceutical composition to a patient, and a hand-held auto-injector adapted for intramuscular or subcutaneous delivery of a pharmaceutical composition to a patient.

In certain embodiments disclosed herein, including in any of paragraphs [034]-[036], the co-packaged drug product further comprises protective packaging.

In certain embodiments disclosed herein, including in any of paragraphs [034]-[037], the co-packaged drug product further comprises instructions describing use of said opioid agonist.

In certain embodiments disclosed herein, including in any of paragraphs [034]-[038], the co-packaged drug product further comprises instructions describing use of said opioid antagonist.

In certain embodiments disclosed herein, including in any of paragraphs [034]-[039], said opioid antagonist is for treating opioid overdose or suspected opioid overdose.

In certain embodiments disclosed herein, including in any of paragraphs [034]-[040], the co-packaged drug product further comprises printed matter describing the use of said opioid antagonist to treat opioid overdose or suspected opioid overdose.

In certain embodiments disclosed herein, including in any of paragraphs [034]-[041], the co-packaged drug product further comprises further comprising a pre-recorded media device describing the use of said opioid antagonist to treat opioid overdose or suspected opioid overdose.

In certain embodiments disclosed herein, including in any of paragraphs [034]-[042], the co-packaged drug product further comprises instructions for downloading an application to a mobile electronic device, wherein the application enables the use of said opioid antagonist to treat opioid overdose or suspected opioid overdose.

In certain embodiments disclosed herein, including in any of paragraphs [034]-[043], said drug delivery device is a hand-held auto-injector adapted for intramuscular or subcutaneous delivery of a pharmaceutical composition to a patient, and said therapeutically effective amount of said opioid antagonist is equivalent to about 0.02 mg to about 2 mg of naloxone hydrochloride.

In certain embodiments disclosed herein, including in any of paragraphs [034]-[044], said drug delivery device is a hand-held auto-injector, and said therapeutically effective amount of said opioid antagonist is equivalent to about 0.4 mg of naloxone hydrochloride.

In certain embodiments disclosed herein, including in any of paragraphs [034]-[045], said drug delivery device is a pre-primed device adapted for nasal delivery of a pharmaceutical composition to a patient; and wherein said therapeutically effective amount of said opioid antagonist is equivalent to about 2 mg to about 12 mg of naloxone hydrochloride.

In certain embodiments disclosed herein, including in any of paragraphs [034]-[046], said therapeutically effective amount of said opioid antagonist is equivalent to about 2 mg of naloxone hydrochloride.

In certain embodiments disclosed herein, including in any of paragraphs [034]-[047], said therapeutically effective amount of said opioid antagonist is equivalent to about 4 mg to about 10 mg of naloxone hydrochloride.

In certain embodiments disclosed herein, including in any of paragraphs [034]-[048], said therapeutically effective amount of said opioid antagonist is equivalent to about 4 mg of naloxone hydrochloride.

In certain embodiments disclosed herein, including in any of paragraphs [034]-[049], said therapeutically effective amount of said opioid antagonist is equivalent to about 8 mg of naloxone hydrochloride.

In certain embodiments disclosed herein, including in any of paragraphs [034]-[050], said patient is in a lying, supine, or recovery position. In certain embodiments disclosed herein, including in any of paragraphs [034]-[0], said patient is in a lying position. In certain embodiments disclosed herein, including in any of paragraphs [034]-[0], said patient is in a supine position.

In certain embodiments disclosed herein, including in any of paragraphs [034]-[051], said patient is in a recovery position.

In certain embodiments disclosed herein, including in any of paragraphs [034]-[052], said naloxone is naloxone hydrochloride.

In certain embodiments disclosed herein, including in any of paragraphs [034]-[053], said naloxone hydrochloride is formulated in a pharmaceutical composition which is a solution.

In certain embodiments disclosed herein, including in any of paragraphs [034]-[054], said pharmaceutical composition further comprises one or more excipients selected from water, NaCl, benzalkonium chloride, sodium edetate, disodium edetate, and hydrochloric acid.

In certain embodiments disclosed herein, including in any of paragraphs [034]-[055], said pharmaceutical composition is substantially free of antimicrobial preservatives.

In certain embodiments disclosed herein, including in any of paragraphs [034]-[056], said pharmaceutical composition further comprises water, NaCl, benzalkonium chloride, disodium edetate, and hydrochloric acid.

In certain embodiments disclosed herein, including in any of paragraphs [034]-[057], said pharmaceutical composition comprises: an isotonicity agent; a preservative; a stabilizing agent; an amount of an acid sufficient to achieve a pH or 3.5-5.5; and an amount of water sufficient to achieve a final volume of about 100 μL.

In certain embodiments disclosed herein, including in any of paragraphs [034]-[058], said pharmaceutical composition comprises: between about 0.2 mg and about 1.2 mg of an isotonicity agent; between about 0.005 mg and about 0.015 mg of a preservative; between about 0.01 mg and about 0.05 mg of a stabilizing agent; an amount of an acid sufficient to achieve a pH or 3.5-5.5; and an amount of water sufficient to achieve a final volume of about 100 μL.

In certain embodiments disclosed herein, including in any of paragraphs [034]-[059], the isotonicity agent is NaCl; the preservative is benzalkonium chloride; the stabilizing agent is disodium edetate; and the acid is hydrochloric acid.

In certain embodiments disclosed herein, including in any of paragraphs [034]-[060], said pharmaceutical composition comprises: about 0.74 mg NaCl; about 0.01 mg benzalkonium chloride; about 0.2 mg disodium edetate; an amount of hydrochloric acid sufficient to achieve a pH or 3.5-5.5; and an amount of water sufficient to achieve a final volume of about 100 μL.

In certain embodiments disclosed herein, including in any of paragraphs [034]-[061], said drug delivery device is filled with said pharmaceutical composition using sterile filling.

In certain embodiments disclosed herein, including in any of paragraphs [034]-[062], said pharmaceutical composition is storage-stable for about twelve months at about 25° C. and about 60% relative humidity.

In certain embodiments disclosed herein, including in any of paragraphs [034]-[063], said drug delivery device is a single-dose device, wherein said pharmaceutical composition is present in one reservoir, and wherein said therapeutically effective amount of said opioid antagonist is delivered essentially by one actuation of said drug delivery device into one nostril of said patient.

In certain embodiments disclosed herein, including in any of paragraphs [034]-[064], the volume of said pharmaceutical composition in said reservoir is not more than about 140 μL.

In certain embodiments disclosed herein, including in any of paragraphs [034]-[065], about 100 μL of said pharmaceutical composition in said reservoir is delivered to said patient in one actuation.

In certain embodiments disclosed herein, including in any of paragraphs [034]-[066], said drug delivery device is a bi-dose device, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device into a first nostril of said patient and a second actuation of said drug delivery device into a second nostril of said patient.

In certain embodiments disclosed herein, including in any of paragraphs [034]-[067], said first volume and said second volume combined is equal to not more than about 380 μL.

In certain embodiments disclosed herein, including in any of paragraphs [034]-[068], about 100 μL of said first volume of said pharmaceutical composition is delivered by said first actuation.

In certain embodiments disclosed herein, including in any of paragraphs [034]-[069], about 100 μL of said second volume of said pharmaceutical composition is delivered by said second actuation.

In certain embodiments disclosed herein, including in any of paragraphs [034]-[070], said drug delivery device is actuatable with one hand.

In certain embodiments disclosed herein, including in any of paragraphs [034]-[071], the delivery time is less than about 25 seconds.

In certain embodiments disclosed herein, including in any of paragraphs [034]-[072], the delivery time is less than about 20 seconds.

In certain embodiments disclosed herein, including in any of paragraphs [034]-[073], the 90% confidence interval for dose delivered per actuation is ± about 2%. In certain embodiments disclosed herein, including in any of paragraphs [034]-[073], the 95% confidence interval for dose delivered per actuation is ± about 2.5%. In certain embodiments disclosed herein, including in any of paragraphs [034]-[073], the 99% confidence interval for dose delivered per actuation is ± about 3%.

In certain embodiments disclosed herein, including in any of paragraphs [034]-[074], upon nasal delivery of said pharmaceutical composition to said patient, less than about 20% of said pharmaceutical composition leaves the nasal cavity via drainage into the nasopharynx or externally. In certain embodiments disclosed herein, including in any of paragraphs [034]-[074], upon nasal delivery of said pharmaceutical composition to said patient, less than about 10% of said pharmaceutical composition leaves the nasal cavity via drainage into the nasopharynx or externally. In certain embodiments disclosed herein, including in any of paragraphs [034]-[074], upon nasal delivery of said pharmaceutical composition to said patient, less than about 5% of said pharmaceutical composition leaves the nasal cavity via drainage into the nasopharynx or externally.

In certain embodiments disclosed herein, including in any of paragraphs [034]-[075], the plasma concentration versus time curve of said opioid antagonist in said patient has a Tmax of less than 30 minutes. In certain embodiments disclosed herein, including in any of paragraphs [034]-[075], the plasma concentration versus time curve of said opioid antagonist in said patient has a Tmax of less than 25 minutes. In certain embodiments disclosed herein, including in any of paragraphs [034]-[075], the plasma concentration versus time curve of said opioid antagonist in said patient has a Tmax of about 20 minutes.

In certain embodiments disclosed herein, including in any of paragraphs [034]-[076], delivery of said therapeutically effective amount of said opioid antagonist to said patient, provides occupancy at Tmax of said opioid antagonist at the opioid receptors in the respiratory control center of said patient of greater than about 90%. In certain embodiments disclosed herein, including in any of paragraphs [034]-[76], delivery of said therapeutically effective amount of said opioid antagonist to said patient, provides occupancy at Tmax of said opioid antagonist at the opioid receptors in the respiratory control center of said patient of greater than about 95%. In certain embodiments disclosed herein, including in any of paragraphs [034]-[076], delivery of said therapeutically effective amount of said opioid antagonist to said patient, provides occupancy at Tmax of said opioid antagonist at the opioid receptors in the respiratory control center of said patient of greater than about 99%.

In certain embodiments disclosed herein, including in any of paragraphs [034]-[077], said patient is free from respiratory depression for at least about 1 hour following treatment consisting essentially of delivery of said therapeutically effective amount of said opioid antagonist. In certain embodiments disclosed herein, including in any of paragraphs [034]-[077], said patient is free from respiratory depression for at least about 2 hours following treatment consisting essentially of delivery of said therapeutically effective amount of said opioid antagonist. In certain embodiments disclosed herein, including in any of paragraphs [034]-[077], said patient is free from respiratory depression for at least about 4 hours following treatment consisting essentially of delivery of said therapeutically effective amount of said opioid antagonist. In certain embodiments disclosed herein, including in any of paragraphs [034]-[077], said patient is free from respiratory depression for at least about 6 hours following treatment consisting essentially of delivery of said therapeutically effective amount of said opioid antagonist.

In certain embodiments disclosed herein, including in any of paragraphs [034]-[078], said therapeutically effective amount of said opioid antagonist is delivered by an untrained individual.

In certain embodiments disclosed herein, including in any of paragraphs [034]-[079], said opioid antagonist is the only pharmaceutically active compound in said delivery device.

In certain embodiments disclosed herein, including in any of paragraphs [034]-[080], said opioid antagonist is naloxone hydrochloride.

In certain embodiments disclosed herein, including in any of paragraphs [034]-[081], the patient exhibits one or more symptoms chosen from: respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In certain embodiments disclosed herein, including in any of paragraphs [034]-[081], the patient exhibits respiratory depression. In certain embodiments disclosed herein, including in any of paragraphs [034]-[081], said respiratory depression is caused by the illicit use of opioids, or by an accidental misuse of opioids during medical opioid therapy.

In certain embodiments disclosed herein, including in any of paragraphs [034]-[082], said patient is free from respiratory depression for at least about 1 hour following treatment consisting essentially of delivery of said therapeutically effective amount of said opioid antagonist. In certain embodiments disclosed herein, including in any of paragraphs [034]-[082], said patient is free from respiratory depression for at least about 2 hours following treatment consisting essentially of delivery of said therapeutically effective amount of said opioid antagonist. In certain embodiments disclosed herein, including in any of paragraphs [034]-[082], said patient is free from respiratory depression for at least about 4 hours following treatment consisting essentially of delivery of said therapeutically effective amount of said opioid antagonist. In certain embodiments disclosed herein, including in any of paragraphs [034]-[082], said patient is free from respiratory depression for at least about 6 hours following treatment consisting essentially of delivery of said therapeutically effective amount of said opioid antagonist.

In certain embodiments disclosed herein, including in any of paragraphs [034]-[083], said opioid antagonist is for use in the treatment of an opioid overdose symptom selected from: respiratory depression, altered level consciousness, miotic pupils, cardiovascular depression, hypoxemia, acute lung injury, aspiration pneumonia, sedation, and hypotension.

Also provided herein is a drug product comprising a combination of a therapeutically effective amount of an opioid agonist and a therapeutically effective amount of naloxone hydrochloride or a hydrate thereof, wherein said naloxone hydrochloride or hydrate thereof is contained in a single-use, pre-primed device adapted for nasal delivery of a pharmaceutical composition to a patient by one actuation of said device into one nostril of said patient, and wherein the single-use, pre-primed device comprises a reservoir containing a pharmaceutical composition which is an aqueous solution of about 100 μL comprising: between about 2 mg and about 12 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [034]-[085], the single-use, pre-primed device adapted for nasal delivery of a pharmaceutical composition to a patient comprises about 2 mg, about 4 mg, or about 8 mg of the naloxone hydrochloride or a hydrate thereof.

Also provided herein is a co-packaged drug product comprising a therapeutically effective amount of an opioid agonist, and a therapeutically effective amount of naloxone hydrochloride or a hydrate thereof; wherein said naloxone hydrochloride or hydrate thereof is contained in a single-use, pre-primed device adapted for nasal delivery of a pharmaceutical composition to a patient by one actuation of said device into one nostril of said patient, having a single reservoir comprising a pharmaceutical composition which is an aqueous solution of about 100 μL comprising: about 4 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [034]-[087]: the isotonicity agent is NaCl; the preservative is benzalkonium chloride; the stabilizing agent is disodium edetate; and the acid is hydrochloric acid.

In certain embodiments disclosed herein, including in any of paragraphs [034]-[088], the aqueous solution comprises: about 4.4 mg naloxone hydrochloride dihydrate; about 0.74 mg NaCl; about 0.01 mg benzalkonium chloride; about 0.2 mg disodium edetate; and an amount of hydrochloric acid sufficient to achieve a pH or 3.5-5.5.

Also provided herein is a drug product comprising a combination of a therapeutically effective amount of an opioid agonist and a therapeutically effective amount of naloxone hydrochloride or a hydrate thereof, wherein said naloxone hydrochloride or hydrate thereof is contained in a pre-primed, bi-dose device adapted for nasal delivery of a pharmaceutical composition to a patient, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device from said first reservoir into a nostril of said patient and a second actuation of said drug delivery device from said second reservoir into a nostril of said patient; each reservoir comprising a pharmaceutical composition which is an aqueous solution of about 100 μL comprising: between about 2 mg and about 12 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [034]-[090], each reservoir of the pre-primed, bi-dose device adapted for nasal delivery of a pharmaceutical composition to a patient comprises about 2 mg, about 4 mg, or about 8 mg of the naloxone hydrochloride or a hydrate thereof.

Also provided herein is a co-packaged drug product comprising a therapeutically effective amount of an opioid agonist, and a therapeutically effective amount of naloxone hydrochloride or a hydrate thereof; wherein said naloxone hydrochloride or hydrate thereof is contained in a pre-primed, bi-dose device adapted for nasal delivery of a pharmaceutical composition to a patient, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device from said first reservoir into a nostril of said patient and a second actuation of said drug delivery device from said second reservoir into a nostril of said patient; each reservoir comprising a pharmaceutical composition which is an aqueous solution of about 100 μL comprising: about 2 mg or about 4 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [034]-[092], each reservoir comprises about 2 mg of the naloxone hydrochloride or a hydrate thereof. The In certain embodiments disclosed herein, including in any of paragraphs [034]-[092], each reservoir comprises about 4 mg of the naloxone hydrochloride or a hydrate thereof.

In certain embodiments disclosed herein, including in any of paragraphs [034]-[093]: the isotonicity agent is NaCl; the preservative is benzalkonium chloride; the stabilizing agent is disodium edetate; and the acid is hydrochloric acid.

In certain embodiments disclosed herein, including in any of paragraphs [034]-[094], the aqueous solution comprises: about 2.2 mg or about 4.4 mg naloxone hydrochloride dihydrate; about 0.74 mg NaCl; about 0.01 mg benzalkonium chloride; about 0.2 mg disodium edetate; and an amount of hydrochloric acid sufficient to achieve a pH or 3.5-5.5.

In certain embodiments disclosed herein, including in any of paragraphs [034]-[095], each reservoir comprises about 2.2 mg of the naloxone hydrochloride dihydrate. In certain embodiments disclosed herein, including in any of paragraphs [034]-[095], each reservoir comprises about 4.4 mg of the naloxone hydrochloride dihydrate.

In certain embodiments disclosed herein, including in any of paragraphs [034]-[096], said opioid antagonist is for use in the emergency treatment of known or suspected opioid overdose, as manifested by one or more symptoms selected from respiratory depression and central nervous system depression.

In certain embodiments disclosed herein, including in any of paragraphs [034]-[097], said opioid antagonist is for use in the emergency treatment of known or suspected opioid overdose characterized by one or more symptoms selected from decreased breathing rate, decreased heart rate, and loss of consciousness.

In certain embodiments disclosed herein, including in any of paragraphs [034]-[099], said symptom is caused by misuse of said opioid agonist.

In certain embodiments disclosed herein, including in any of paragraphs [034]-[099], said symptom is respiratory depression.

In certain embodiments disclosed herein, including in any of paragraphs [034]-[0100], said opioid antagonist is for use in the complete or partial reversal of narcotic depression, including respiratory depression, induced by opioids selected from: natural and synthetic narcotics, propoxyphene, methadone, nalbuphine, pentazocine and butorphanol.

Also provided herein is a method of lowering opioid overdose risk in an individual at risk for opioid overdose, comprising providing to the individual at risk for opioid overdose a combination of a therapeutically effective amount of an opioid agonist and a therapeutically effective amount of naloxone hydrochloride or a hydrate thereof, wherein said naloxone hydrochloride or hydrate thereof is contained in a drug delivery device selected from a pre-primed device adapted for nasal delivery of a pharmaceutical composition to a patient by one actuation of said device into one nostril of said patient, and a hand-held auto-injector adapted for intramuscular or subcutaneous delivery of a pharmaceutical composition to a patient.

In certain embodiments disclosed herein, including in paragraph [0102], the opioid agonist and opioid antagonist are provided contemporaneously. In certain embodiments disclosed herein, including in any of paragraphs [0102]-[097], the opioid agonist and opioid antagonist are provided sequentially. In certain embodiments disclosed herein, including in paragraph [0102], the opioid agonist and opioid antagonist are provided during a single visit with a provider. In certain embodiments disclosed herein, including in paragraph [0102], the opioid agonist and opioid antagonist are co-packaged.

Also provided herein is a method of lowering opioid overdose risk in an individual at risk for opioid overdose, comprising providing to the individual at risk for opioid overdose a co-packaged drug product comprising a therapeutically effective amount of an opioid agonist, and a therapeutically effective amount of an opioid antagonist selected from naloxone and pharmaceutically acceptable salts thereof; wherein said opioid antagonist is contained in a drug delivery device selected from a pre-primed device adapted for nasal delivery of a pharmaceutical composition to a patient, and a hand-held auto-injector adapted for intramuscular or subcutaneous delivery of a pharmaceutical composition to a patient.

In certain embodiments disclosed herein, including in any of paragraphs [0102]-[0104], said individual at risk for opioid overdose is prescribed the co-packaged drug product, and further comprising counseling said individual or a second individual to whom the product is dispensed on the use of said co-packaged drug product.

In certain embodiments disclosed herein, including in any of paragraphs [0102]-[0105], said co-packaged drug product further comprises protective packaging.

In certain embodiments disclosed herein, including in any of paragraphs [0102]-[0106], said co-packaged drug product further comprises instructions describing use of said opioid agonist.

In certain embodiments disclosed herein, including in any of paragraphs [0102]-[0107], said co-packaged drug product further comprises instructions describing use of said opioid antagonist.

In certain embodiments disclosed herein, including in any of paragraphs [0102]-[01108], said opioid antagonist is for treating opioid overdose or suspected opioid overdose.

In certain embodiments disclosed herein, including in any of paragraphs [0102]-[0109], said co-packaged drug product further comprises printed matter describing the use of said opioid antagonist to treat opioid overdose or suspected opioid overdose.

In certain embodiments disclosed herein, including in any of paragraphs [0102]-[0110], said co-packaged drug product further comprises a pre-recorded media device describing the use of said opioid antagonist to treat opioid overdose or suspected opioid overdose.

In certain embodiments disclosed herein, including in any of paragraphs [0102]-[0111], said co-packaged drug product further comprises instructions for downloading an application to a mobile electronic device, wherein the application enables the use of said opioid antagonist to treat opioid overdose or suspected opioid overdose.

In certain embodiments disclosed herein, including in any of paragraphs [0102]-[0112], said drug delivery device is a hand-held auto-injector adapted for intramuscular or subcutaneous delivery of a pharmaceutical composition to a patient, and said therapeutically effective amount of said opioid antagonist is equivalent to about 0.02 mg to about 2 mg of naloxone hydrochloride.

In certain embodiments disclosed herein, including in any of paragraphs [0102]-[0113], said drug delivery device is a hand-held auto-injector, and said therapeutically effective amount of said opioid antagonist is equivalent to about 0.4 mg of naloxone hydrochloride.

In certain embodiments disclosed herein, including in any of paragraphs [0102]-[0114], said drug delivery device is a pre-primed device adapted for nasal delivery of a pharmaceutical composition to a patient, and said therapeutically effective amount of said opioid antagonist is equivalent to about 2 mg to about 12 mg of naloxone hydrochloride. In certain embodiments disclosed herein, including in any of paragraphs [0102]-[0114], said therapeutically effective amount of said opioid antagonist is equivalent to about 2 mg of naloxone hydrochloride. In certain embodiments disclosed herein, including in any of paragraphs [0102]-[0114], said therapeutically effective amount of said opioid antagonist is equivalent to about 4 mg to about 10 mg of naloxone hydrochloride. In certain embodiments disclosed herein, including in any of paragraphs [0102]-[0114], said therapeutically effective amount of said opioid antagonist is equivalent to about 4 mg of naloxone hydrochloride. In certain embodiments disclosed herein, including in any of paragraphs [0102]-[0114], said therapeutically effective amount of said opioid antagonist is equivalent to about 8 mg of naloxone hydrochloride.

In certain embodiments disclosed herein, including in any of paragraphs [0102]-[0115], said patient is in a lying, supine, or recovery position. In certain embodiments disclosed herein, including in any of paragraphs [0102]-[0115], said patient is in a lying position. In certain embodiments disclosed herein, including in any of paragraphs [0102]-[0115], said patient is in a supine position. In certain embodiments disclosed herein, including in any of paragraphs [0102]-[0115], said patient is in a recovery position.

In certain embodiments disclosed herein, including in any of paragraphs [0102]-[0115], said naloxone is naloxone hydrochloride.

In certain embodiments disclosed herein, including in any of paragraphs [0102]-[0117], said naloxone hydrochloride is formulated in a pharmaceutical composition which is a solution.

In certain embodiments disclosed herein, including in any of paragraphs [0102]-[0118], said pharmaceutical composition further comprises one or more excipients selected from water, NaCl, benzalkonium chloride, sodium edetate, disodium edetate, and hydrochloric acid.

In certain embodiments disclosed herein, including in any of paragraphs [0102]-[0119], said pharmaceutical composition is substantially free of antimicrobial preservatives.

In certain embodiments disclosed herein, including in any of paragraphs [0102]-[0120], said pharmaceutical composition further comprises water, NaCl, benzalkonium chloride, disodium edetate, and hydrochloric acid.

In certain embodiments disclosed herein, including in any of paragraphs [0102]-[0121], said pharmaceutical composition further comprises: an isotonicity agent; a preservative; a stabilizing agent; an amount of an acid sufficient to achieve a pH or 3.5-5.5; and an amount of water sufficient to achieve a final volume of about 100 μL.

In certain embodiments disclosed herein, including in any of paragraphs [0102]-[0122], said pharmaceutical composition comprises: between about 0.2 mg and about 1.2 mg of an isotonicity agent; between about 0.005 mg and about 0.015 mg of a preservative; between about 0.01 mg and about 0.05 mg of a stabilizing agent; an amount of an acid sufficient to achieve a pH or 3.5-5.5; and an amount of water sufficient to achieve a final volume of about 100 μL.

In certain embodiments disclosed herein, including in any of paragraphs [0102]-[0123], the isotonicity agent is NaCl; the preservative is benzalkonium chloride; the stabilizing agent is disodium edetate; and the acid is hydrochloric acid.

In certain embodiments disclosed herein, including in any of paragraphs [0102]-[0124], said pharmaceutical composition comprises: about 0.74 mg NaCl; about 0.01 mg benzalkonium chloride; about 0.2 mg disodium edetate; an amount of hydrochloric acid sufficient to achieve a pH or 3.5-5.5; and an amount of water sufficient to achieve a final volume of about 100 μL.

In certain embodiments disclosed herein, including in any of paragraphs [0102]-[0125], said drug delivery device is filled with said pharmaceutical composition using sterile filling.

In certain embodiments disclosed herein, including in any of paragraphs [0102]-[0126], herein said pharmaceutical composition is storage-stable for about twelve months at about 25° C. and about 60% relative humidity.

In certain embodiments disclosed herein, including in any of paragraphs [0102]-[0127], said drug delivery device is a single-dose device, wherein said pharmaceutical composition is present in one reservoir, and wherein said therapeutically effective amount of said opioid antagonist is delivered essentially by one actuation of said drug delivery device into one nostril of said patient.

In certain embodiments disclosed herein, including in any of paragraphs [0102]-[0128], the volume of said pharmaceutical composition in said reservoir is not more than about 140 μL.

In certain embodiments disclosed herein, including in any of paragraphs [0102]-[0129], about 100 μL of said pharmaceutical composition in said reservoir is delivered to said patient in one actuation.

In certain embodiments disclosed herein, including in any of paragraphs [0102]-[0130], said drug delivery device is a bi-dose device, wherein a first volume of said pharmaceutical composition is present in a first reservoir and a second volume of said pharmaceutical composition is present in a second reservoir, and wherein said therapeutically effective amount of said opioid antagonist is delivered essentially by a first actuation of said drug delivery device into a first nostril of said patient and a second actuation of said drug delivery device into a second nostril of said patient.

In certain embodiments disclosed herein, including in any of paragraphs [0102]-[0131], said first volume and said second volume combined is equal to not more than about 380 μL. In certain embodiments disclosed herein, including in any of paragraphs [0102]-[0131], about 100 μL of said first volume of said pharmaceutical composition is delivered by said first actuation. In certain embodiments disclosed herein, including in any of paragraphs [0102]-[0131], about 100 μL of said second volume of said pharmaceutical composition is delivered by said second actuation.

In certain embodiments disclosed herein, including in any of paragraphs [0102]-[0132], said drug delivery device is actuatable with one hand.

In certain embodiments disclosed herein, including in any of paragraphs [0102]-[0133], the delivery time is less than about 25 seconds. In certain embodiments disclosed herein, including in any of paragraphs [0102]-[0125], the delivery time is less than about 20 seconds.

In certain embodiments disclosed herein, including in any of paragraphs [0102]-[0134], the 90% confidence interval for dose delivered per actuation is ± about 2%. In certain embodiments disclosed herein, including in any of paragraphs [0102]-[0134], the 95% confidence interval for dose delivered per actuation is ± about 2.5%. In certain embodiments disclosed herein, including in any of paragraphs [0102]-[0134], the 99% confidence interval for dose delivered per actuation is ± about 3%.

In certain embodiments disclosed herein, including in any of paragraphs [0102]-[0135], upon nasal delivery of said pharmaceutical composition to said patient, less than about 20% of said pharmaceutical composition leaves the nasal cavity via drainage into the nasopharynx or externally. In certain embodiments disclosed herein, including in any of paragraphs [0102]-[0135], upon nasal delivery of said pharmaceutical composition to said patient, less than about 10% of said pharmaceutical composition leaves the nasal cavity via drainage into the nasopharynx or externally. In certain embodiments disclosed herein, including in any of paragraphs [0102]-[0135], upon nasal delivery of said pharmaceutical composition to said patient, less than about 5% of said pharmaceutical composition leaves the nasal cavity via drainage into the nasopharynx or externally.

In certain embodiments disclosed herein, including in any of paragraphs [0102]-[0136], the plasma concentration versus time curve of said opioid antagonist in said patient has a Tmax of less than 30 minutes. In certain embodiments disclosed herein, including in any of paragraphs [0102]-[0136], the plasma concentration versus time curve of said opioid antagonist in said patient has a Tmax of less than 25 minutes. In certain embodiments disclosed herein, including in any of paragraphs [0102]-[0136], the plasma concentration versus time curve of said opioid antagonist in said patient has a Tmax of about 20 minutes.

In certain embodiments disclosed herein, including in any of paragraphs [0102]-[0137], delivery of said therapeutically effective amount of said opioid antagonist to said patient, provides occupancy at Tmax of said opioid antagonist at the opioid receptors in the respiratory control center of said patient of greater than about 90%. In certain embodiments disclosed herein, including in any of paragraphs [0102]-[0137], delivery of said therapeutically effective amount of said opioid antagonist to said patient, provides occupancy at Tmax of said opioid antagonist at the opioid receptors in the respiratory control center of said patient of greater than about 95%.

In certain embodiments disclosed herein, including in any of paragraphs [0102]-[0138], delivery of said therapeutically effective amount of said opioid antagonist to said patient, provides occupancy at Tmax of said opioid antagonist at the opioid receptors in the respiratory control center of said patient of greater than about 99%.

In certain embodiments disclosed herein, including in any of paragraphs [0102]-[0139], said patient is free from respiratory depression for at least about 1 hour following treatment consisting essentially of delivery of said therapeutically effective amount of said opioid antagonist. In certain embodiments disclosed herein, including in any of paragraphs [0102]-[0139], said patient is free from respiratory depression for at least about 2 hours following treatment consisting essentially of delivery of said therapeutically effective amount of said opioid antagonist. In certain embodiments disclosed herein, including in any of paragraphs [0102]-[0139], said patient is free from respiratory depression for at least about 4 hours following treatment consisting essentially of delivery of said therapeutically effective amount of said opioid antagonist. In certain embodiments disclosed herein, including in any of paragraphs [0102]-[0139], said patient is free from respiratory depression for at least about 6 hours following treatment consisting essentially of delivery of said therapeutically effective amount of said opioid antagonist. In certain embodiments disclosed herein, including in any of paragraphs [0102]-[0139], said therapeutically effective amount of said opioid antagonist is delivered by an untrained individual.

In certain embodiments disclosed herein, including in any of paragraphs [0102]-[0140], said opioid antagonist is the only pharmaceutically active compound in said delivery device.

In certain embodiments disclosed herein, including in any of paragraphs [0102]-[0141], said opioid antagonist is naloxone hydrochloride.

In certain embodiments disclosed herein, including in any of paragraphs [0102]-[0142], the patient exhibits one or more symptoms chosen from: respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In certain embodiments disclosed herein, including in any of paragraphs [0102]-[0134], the patient exhibits respiratory depression.

In certain embodiments disclosed herein, including in any of paragraphs [0102]-[0143], said respiratory depression is caused by the illicit use of opioids, or by an accidental misuse of opioids during medical opioid therapy.

Also provided herein is a method of lowering opioid overdose risk in an individual at risk for opioid overdose, comprising providing to the individual at risk for opioid overdose a combination of a therapeutically effective amount of an opioid agonist and a therapeutically effective amount of naloxone hydrochloride or a hydrate thereof, wherein said naloxone hydrochloride or hydrate thereof is contained in a single-use, pre-primed device adapted for nasal delivery of a pharmaceutical composition to a patient by one actuation of said device into one nostril of said patient, and wherein the single-use, pre-primed device comprises a reservoir containing a pharmaceutical composition which is an aqueous solution of about 100 μL comprising: between about 2 mg and about 12 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [0102]-[0145], the single-use, pre-primed device adapted for nasal delivery of a pharmaceutical composition to a patient comprises about 2 mg, about 4 mg, or about 8 mg of the naloxone hydrochloride or a hydrate thereof.

Also provided herein is a method of lowering opioid overdose risk in an individual at risk for opioid overdose, comprising providing a co-packaged drug product comprising a therapeutically effective amount of an opioid agonist, and a therapeutically effective amount of naloxone hydrochloride or a hydrate thereof; wherein said naloxone hydrochloride or hydrate thereof is contained in a single-use, pre-primed device adapted for nasal delivery of a pharmaceutical composition to a patient by one actuation of said device into one nostril of said patient, having a single reservoir comprising a pharmaceutical composition which is an aqueous solution of about 100 μL comprising: about 4 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [0102]-[0147]: the isotonicity agent is NaCl; the preservative is benzalkonium chloride; the stabilizing agent is disodium edetate; and the acid is hydrochloric acid.

In certain embodiments disclosed herein, including in any of paragraphs [0102]-[0148], the aqueous solution comprises: about 4.4 mg naloxone hydrochloride dihydrate; about 0.74 mg NaCl; about 0.01 mg benzalkonium chloride; about 0.2 mg disodium edetate; and an amount of hydrochloric acid sufficient to achieve a pH or 3.5-5.5.

Also provided herein is a method of lowering opioid overdose risk in an individual at risk for opioid overdose, comprising providing to the individual at risk for opioid overdose a combination of a therapeutically effective amount of an opioid agonist and a therapeutically effective amount of naloxone hydrochloride or a hydrate thereof, wherein said naloxone hydrochloride or hydrate thereof is contained in a pre-primed, bi-dose device adapted for nasal delivery of a pharmaceutical composition to a patient, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device from said first reservoir into a nostril of said patient and a second actuation of said drug delivery device from said second reservoir into a nostril of said patient; each reservoir comprising a pharmaceutical composition which is an aqueous solution of about 100 μL comprising: between about 2 mg and about 12 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [0102]-[084], each reservoir of the single-use, pre-primed device adapted for nasal delivery of a pharmaceutical composition to a patient comprises about 2 mg, about 4 mg, or about 8 mg of the naloxone hydrochloride or a hydrate thereof.

Also provided herein is a method of lowering opioid overdose risk in an individual at risk for opioid overdose, comprising providing a co-packaged drug product comprising a therapeutically effective amount of an opioid agonist, and a therapeutically effective amount of naloxone hydrochloride or a hydrate thereof; wherein said naloxone hydrochloride or hydrate thereof is contained in a pre-primed, bi-dose device adapted for nasal delivery of a pharmaceutical composition to a patient, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device from said first reservoir into a nostril of said patient and a second actuation of said drug delivery device from said second reservoir into a nostril of said patient; each reservoir comprising a pharmaceutical composition which is an aqueous solution of about 100 μL comprising: about 2 mg or about 4 mg of the naloxone hydrochloride or a hydrate thereof; between about 0.2 mg and about 1.2 mg of an isotonicity agent; between about 0.005 mg and about 0.015 mg of a preservative; between about 0.01 mg and about 0.05 mg of a stabilizing agent; and an amount of acid sufficient to achieve a pH or 3.5-5.5.

In certain embodiments disclosed herein, including in any of paragraphs [0102]-[0152], each reservoir comprises about 2 mg of the naloxone hydrochloride or a hydrate thereof. In certain embodiments disclosed herein, including in any of paragraphs [0102]-[0152], each reservoir comprises about 4 mg of the naloxone hydrochloride or a hydrate thereof.

In certain embodiments disclosed herein, including in any of paragraphs [0102]-[0153]: the isotonicity agent is NaCl; the preservative is benzalkonium chloride; the stabilizing agent is disodium edetate; and the acid is hydrochloric acid.

In certain embodiments disclosed herein, including in any of paragraphs [0102]-[0154], the aqueous solution comprises: about 2.2 mg or about 4.4 mg naloxone hydrochloride dihydrate; about 0.74 mg NaCl; about 0.01 mg benzalkonium chloride; about 0.2 mg disodium edetate; and an amount of hydrochloric acid sufficient to achieve a pH or 3.5-5.5.

In certain embodiments disclosed herein, including in any of paragraphs [0102]-[0155], each reservoir comprises about 2.2 mg of the naloxone hydrochloride dihydrate. In certain embodiments disclosed herein, including in any of paragraphs [0102]-[0155], each reservoir comprises about 4.4 mg of the naloxone hydrochloride dihydrate.

In certain embodiments disclosed herein, including in any of paragraphs [0102]-[0156], said opioid antagonist is for use in the treatment of an opioid overdose symptom selected from: respiratory depression, altered level consciousness, miotic pupils, cardiovascular depression, hypoxemia, acute lung injury, aspiration pneumonia, sedation, and hypotension.

In certain embodiments disclosed herein, including in any of paragraphs [0102]-[1457], said opioid antagonist is for use in the emergency treatment of known or suspected opioid overdose, as manifested by one or more symptoms selected from: respiratory depression and central nervous system depression.

In certain embodiments disclosed herein, including in any of paragraphs [0102]-[0158], said opioid antagonist is for use in the emergency treatment of known or suspected opioid overdose characterized by one or more symptoms selected from: decreased breathing rate, decreased heart rate, and loss of consciousness.

In certain embodiments disclosed herein, including in any of paragraphs [0102]-[0159], said symptom is caused by misuse of said opioid agonist.

In certain embodiments disclosed herein, including in any of paragraphs [0102]-[0160], said symptom is respiratory depression.

In certain embodiments disclosed herein, including in any of paragraphs [0102]-[0161], said opioid antagonist is for use in the complete or partial reversal of narcotic depression, including respiratory depression, induced by opioids selected from: natural and synthetic narcotics, propoxyphene, methadone, nalbuphine, pentazocine and butorphanol.

In certain embodiments disclosed herein, including in any of paragraphs [034]-[0162], said opioid agonist is selected from alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof. In certain embodiments disclosed herein, including in any of paragraphs [034]-[0162], said opioid agonist is selected from alfentanil and pharmaceutically acceptable salts thereof. In certain embodiments disclosed herein, including in any of paragraphs [034]-[0162], said opioid agonist is selected from buprenorphine and pharmaceutically acceptable salts thereof. In certain embodiments disclosed herein, including in any of paragraphs [034]-[0162], said opioid agonist is selected from butorphanol and pharmaceutically acceptable salts thereof. In certain embodiments disclosed herein, including in any of paragraphs [034]-[0162], said opioid agonist is selected from codeine and pharmaceutically acceptable salts thereof. In certain embodiments disclosed herein, including in any of paragraphs [034]-[0162], said opioid agonist is selected from diamorphine and pharmaceutically acceptable salts thereof. In certain embodiments disclosed herein, including in any of paragraphs [034]-[0162], said opioid agonist is selected from dextromoramide and pharmaceutically acceptable salts thereof. In certain embodiments disclosed herein, including in any of paragraphs [034]-[0162], said opioid agonist is selected from dezocine and pharmaceutically acceptable salts thereof. In certain embodiments disclosed herein, including in any of paragraphs [034]-[0162], said opioid agonist is selected from dihydrocodeine and pharmaceutically acceptable salts thereof. In certain embodiments disclosed herein, including in any of paragraphs [034]-[0162], said opioid agonist is selected from fentanyl and pharmaceutically acceptable salts thereof. In certain embodiments disclosed herein, including in any of paragraphs [034]-[0162], said opioid agonist is selected from hydrocodone and pharmaceutically acceptable salts thereof. In certain embodiments disclosed herein, including in any of paragraphs [034]-[0162], said opioid agonist is selected from hydromorphone and pharmaceutically acceptable salts thereof. In certain embodiments disclosed herein, including in any of paragraphs [034]-[0162], said opioid agonist is selected from levorphanol and pharmaceutically acceptable salts thereof. In certain embodiments disclosed herein, including in any of paragraphs [034]-[0162], said opioid agonist is selected from meperidine and pharmaceutically acceptable salts thereof. In certain embodiments disclosed herein, including in any of paragraphs [034]-[0162], said opioid agonist is selected from meptazinol and pharmaceutically acceptable salts thereof. In certain embodiments disclosed herein, including in any of paragraphs [034]-[0162], said opioid agonist is selected from methadone and pharmaceutically acceptable salts thereof. In certain embodiments disclosed herein, including in any of paragraphs [034]-[0162], said opioid agonist is selected from morphine and pharmaceutically acceptable salts thereof. In certain embodiments disclosed herein, including in any of paragraphs [034]-[0162], said opioid agonist is selected from nalbuphine and pharmaceutically acceptable salts thereof. In certain embodiments disclosed herein, including in any of paragraphs [034]-[0162], said opioid agonist is selected from nalorphine and pharmaceutically acceptable salts thereof. In certain embodiments disclosed herein, including in any of paragraphs [034]-[0162], said opioid agonist is opium. In certain embodiments disclosed herein, including in any of paragraphs [034]-[0162], said opioid agonist is selected from oxycodone and pharmaceutically acceptable salts thereof. In certain embodiments disclosed herein, including in any of paragraphs [034]-[0162], said opioid agonist is selected from oxymorphone and pharmaceutically acceptable salts thereof. In certain embodiments disclosed herein, including in any of paragraphs [034]-[0162], said opioid agonist is selected from selected from: pentazocine and pharmaceutically acceptable salts thereof. In certain embodiments disclosed herein, including in any of paragraphs [034]-[0162], said opioid agonist is selected from propoxyphene and pharmaceutically acceptable salts thereof. In certain embodiments disclosed herein, including in any of paragraphs [034]-[0162], said opioid agonist is selected from remifentanyl and pharmaceutically acceptable salts thereof. In certain embodiments disclosed herein, including in any of paragraphs [034]-[0162], said opioid agonist is selected from sufentanyl and pharmaceutically acceptable salts thereof. In certain embodiments disclosed herein, including in any of paragraphs [034]-[0162], said opioid agonist is selected from tapentadol and pharmaceutically acceptable salts thereof. In certain embodiments disclosed herein, including in any of paragraphs [034]-[0150], said opioid agonist is tramadol and pharmaceutically acceptable salts thereof.

Also provided are embodiments wherein any embodiment disclosed herein, and particularly any embodiment disclosed above in paragraphs [034]-[0162] above or in relevant paragraphs below, may be combined with any one or more of these embodiments, provided the combination is not mutually exclusive.

Also provided are uses of the co-packaged drug products as disclosed herein for treatment of diseases, disorders, conditions, and/or symptoms thereof as disclosed herein, corresponding to methods of treatment disclosed above in paragraphs [0102]-[0162].

### DEFINITIONS

For clarity and consistency, the following definitions will be used throughout this patent document.

The term “active ingredient” or “pharmaceutically active compound” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The term “actuation,” as used herein, refers to operation of the drug delivery device such that the pharmaceutical composition is delivered therefrom.

The term “agonist,” as used herein, refers to as used herein refers to a moiety that interacts with and activates a receptor, and thereby initiates a physiological or pharmacological response characteristic of that receptor. The term “antagonist,” as used herein, refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “antimicrobial preservative,” as used herein, refers to a pharmaceutically acceptable excipient with antimicrobial properties which is added to a pharmaceutical composition to maintain microbiological stability.

The term “application”, as used herein, refers to a program executed by a computer.

The term “AUC,” as used herein, refers to the area under the drug plasma concentration-time curve. The term “AUC₀₋ₜ,” as used herein, refers to the area under the drug plasma concentration-time curve from t=0 to the last measurable concentration. The term “AUC_{0-∞}.,” as used herein, refers to the area under the drug plasma concentration-time curve extrapolated to ∞. The term “AUC_{0-t/D},” as used herein, refers to the AUC₀₋ₜ normalized to 0.4 mg IM naloxone. The term “AUC_{0-∞/D},” as used herein, refers to the AUC_{0-∞} normalized to 0.4 mg IM naloxone

The term “bioavailability (F),” as used herein, refers to the fraction of a dose of drug that is absorbed from its site of administration and reaches, in an unchanged form, the systemic circulation. The term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1} }$$

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. The term “apparent clearance (CL/F),” as used herein, refers to clearance that does not take into account the bioavailability of the drug. It is the ratio of the dose over the AUC.

The term “Cₘₐₓ,” as used herein, refers to the maximum observed plasma concentration. The term “C_{max/D},” as used herein, refers to Cₘₐₓ normalized to 0.4 mg IM naloxone.

The term “coefficient of variation (CV),” as used herein, refers to the ratio of the sample standard deviation to the sample mean. It is often expressed as a percentage.

The term “confidence interval,” as used herein, refers to a range of values which will include the true average value of a parameter a specified percentage of the time.

The term “co-packaged drug product”, as used herein, refers to two or more separate drug products in their final dosage form, packaged together. In some embodiments the co-packaged drug product comprises appropriate labeling to support the combination use.

The term “drug delivery device”, as used herein, refers to an apparatus capable of delivering a drug to patient in need thereof.

The term “delivery time,” as used herein, refers to the amount of time that elapses between a determination made by a healthcare professional, or an untrained individual that an individual is in need of nasal delivery of an opioid antagonist and completion of the delivery.

The term “drug product comprising a combination” as used herein refers to a drug product with any physical combination of two or more active ingredients such as, for example, an opioid agonist and an opioid antagonist. In certain embodiments, the two or more active ingredients are separately enclosed in one or more containers. In other embodiments, the two or more active ingredients are arranged in a single package or dispensing device. The drug product optionally comprises directions on how to use the two or more active ingredients suitable for administration to obtain a therapeutic outcome. As used herein, however, a “drug product comprising a combination” does not include a single dosage form comprising two or more active ingredients such as, for example, an opioid agonist and an opioid antagonist as ingredients of the same capsule or tablet.

The term “elimination rate constant (λ),” as used herein, refers to the fractional rate of drug removal from the body. This rate is constant in first-order kinetics and is independent of drug concentration in the body. λ is the slope of the plasma concentration-time line (on a logarithmic y scale). The term “λ_{z}” as used herein, refers to the terminal phase elimination rate constant, wherein the “terminal phase” of the drug plasma concentration-time curve is a straight line when plotted on a semilogarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

The term “equivalent,” as used herein refers to a weight of an opioid antagonist selected from naloxone and pharmaceutically acceptable salts thereof that is equimolar to a specified weight of naloxone hydrochloride. For example, 8 mg of anhydrous naloxone hydrochloride (molecular weight, 363.84) is equivalent to about 7.2 mg of naloxone freebase (molecular weight, 327.37), and to about 8.8 mg of naloxone hydrochloride dihydrate (molecular weight 399.87).

The term “filled,” as used herein, refers to an association between a device and a pharmaceutical composition, for example, when a pharmaceutical composition described herein comprising a therapeutically effective amount of an opioid antagonist is present within a reservoir that forms a part of a device described herein.

The term “hydrate,” as used herein, refers to an opioid antagonist described herein or a salt thereof that further includes a stoichiometric or non-stoichiometric amount of water bound by non-covalent intermolecular forces.

The term “in need of treatment” and the term “in need thereof” when referring to treatment are used interchangeably and refer to a judgment made by a caregiver (e.g. physician, nurse, nurse practitioner, that a patient will benefit from treatment.

An individual “who is at risk for opioid overdose” includes an individual who illicitly uses opioids, on individual who accidentally ingests opioids, and an individual at risk for accidental misuse of opioids during medical opioid therapy.

As used herein, two embodiments are “mutually exclusive” when one is defined to be something which is different than the other. For example, an embodiment wherein the amount of naloxone hydrochloride is specified to be 4 mg is mutually exclusive with an embodiment wherein the amount of naloxone hydrochloride is specified to be 2 mg. However, an embodiment wherein the amount of naloxone hydrochloride is specified to be 4 mg is not mutually exclusive with an embodiment in which less than about 10% of said pharmaceutical composition leaves the nasal cavity via drainage into the nasopharynx or externally.

The term “naloxone,” as used herein, refers to a compound of the following structure:

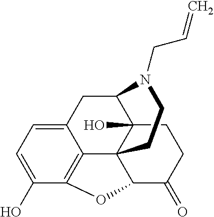

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (−)-17-allyl-4,5α-epoxy-3,14-dihydroxymorphinan-6-one; 4,5α-epoxy-3,14-dihydroxy-17-(2-propenyl)morphinan-6-one; and (−)-12-allyl-7,7a,8,9-tetrahydro-3,7a-dihydroxy-4aH-8,9c-iminoethanophenanthro[4,5-bcd]furan-5(6H)-one. Naloxone hydrochloride may be anhydrous (CAS Reg. No. 357-08-4) and also forms a dihydrate (CAS No. 51481-60-8). It has been sold under various brand names including Narcan®, Nalone®, Nalossone®, Naloxona®, Naloxonum®, Narcanti®, and Narcon®.

The term “naltrexone,” as used herein, refers to a compound of the following structure:

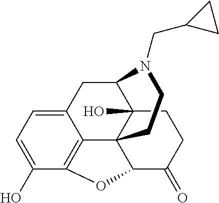

or a pharmaceutically acceptable salt, hydrate, or solvate thereof. The CAS registry number for naltrexone is 16590-41-3. Other names for naltrexone include: 17-(cyclopropylmethyl)-4,5α-epoxy-3,14-dihydroxymorphinan-6-one; (5α)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacyclo[9.6.1.01,13.05,17.07,18]octadeca-7(18),8,10-trien-14-one. Naltrexone hydrochloride (CAS Reg. No. 16676-29-2) has been marketed under the trade names Antaxone®, Depade®, Nalorex®, Revia®, Trexan®, Vivitrex®, and Vivitrol®.

The term “methylnaltrexone,” as used herein, refers to a pharmaceutically acceptable salt comprising the cation (5α)-17-(cyclopropylmethyl)-3,14-dihydroxy-17-methyl-4,5-epoxymorphinanium-17-ium-6-one a compound of the following structure:

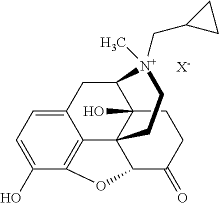

wherein X⁻ is a pharmaceutically acceptable anion. Methylnaltrexone bromide (CAS Reg. No. 75232-52-7) has been marketed under the trade name Relistor®.

The term “nalmefene,” as used herein, refers to 17-cyclopropylmethyl-4,5α-epoxy-6-methylenemorphinan-3,14-diol, a compound of the following structure:

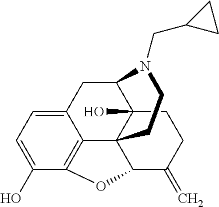

Nalmefene hydrochloride (CAS Reg. No. 58895-64-0) has been marketed under the trade names Nalmetrene®, Cervene®, Revex®, Arthrene®, and Incystene®.

The term “nostril,” as used herein, is synonymous with “naris.”

The term “opioid antagonist” includes, in addition to naloxone and pharmaceutically acceptable salts thereof: naltrexone, methylnaltrexone, and nalmefene, and pharmaceutically acceptable salts thereof. In some embodiments, the opioid antagonist is naloxone hydrochloride. In some embodiments, the opioid antagonist is naloxone hydrochloride dihydrate. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the nasally administering is accomplished using a device described herein.

The term “opioid overdose,” as used herein, refers to an acute medical condition induced by excessive use of one or more opioids. Symptoms of opioid overdose include including respiratory depression (including postoperative opioid respiratory depression, acute lung injury, and aspiration pneumonia), central nervous system depression (which may include sedation, altered level consciousness, miotic (constricted) pupils), and cardiovascular depression (which may include hypoxemia and hypotension). Visible signs of opioid overdose or suspected opioid overdose include: unresponsiveness and/or loss of consciousness (won't respond to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting. Because opioid overdose may be difficult to diagnose and/or quantify, particularly by a lay person, as used herein, treatment of opioid overdose is meant to include treatment of suspected opioid overdose in opioid-intoxicated patients. Opioids that may induce overdose include, codeine, morphine, methadone, fentanyl, oxycodone HCl, hydrocodone bitartrate, hydromorphone, oxymorphone, meperidine, propoxyphene, opium, heroin, tramadol, tapentadol, and certain narcotic-antagonist analgesics, such as, nalbuphine, pentazocine and butorphanol. In some embodiments, the opioid agonist is in a tamper-proof formulation. In some embodiments, the opioid agonist is in a tamper-resistant formulation. In some embodiments, the opioid agonist is selected from Acurox® Oxycodone DETERx®, Egalet hydrocodone, Egalet morphine, Egalet oxycodone, Exalgo®, Opana®, and Remoxy®.

The term “patient,” as used herein, refers to any subject (preferably human) afflicted with a condition likely to benefit from a treatment with a therapeutically effective amount of an opioid antagonist.

The term “pharmaceutical composition,” as used herein, refers to a composition comprising at least one active ingredient; including but not limited to, salts, solvates and hydrates of the opioid antagonists described herein, whereby the composition is amenable to use for a specified, efficacious outcome in a mammal (for example, without limitation, a human).

The term “pre-primed,” as used herein, refers to a drug delivery device, such as a nasal spray which is capable of delivering a pharmaceutical composition to a patient in need thereof with the first actuation of the spray pump, i.e., without the need to prime the pump prior to dosing, such as by actuating the pump one or more times until a spray appears.

The term “prone,” as used herein, refers to a patient who is lying face down.

The term “protective packaging”, as used herein, refers to overwrap.

The term “providing” in the context of providing a co-packaged drug product as disclosed herein to an individual includes co-packaging the drug product, prescribing the co-packaged drug product, and dispensing the co-packaged drug product. The providing may be done either directly to an individual (for example, to an individual for whom an opioid agonist prescription is appropriate, or who is otherwise at risk of opioid overdose) or to a second individual

The term “receptor binding or occupancy” refers to a characterization of the kinetics between a radioactive drug and receptors or other binding sites throughout the body, and characterization of the radioactive drug binding affinity to these receptors.

The term “recovery position,” as used herein, means a position of the human body in which a patient lies on his/her side, with a leg or knee out in front (e.g., to prevent rolling onto his/her stomach) and at least one hand supporting the head (e.g., to elevate the face to facilitate breathing and prevent inhalation of vomit).

The term “solvate,” as used herein, refers to an opioid antagonist described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “sterile filling,” as used herein, refers methods of manufacturing the devices and pharmaceutical compositions described herein, such that the use of preservatives is not required. Sterile drug products may be produced using aseptic processing or terminal sterilization. Terminal sterilization usually involves filling and sealing product containers under high-quality environmental conditions. In an aseptic process, the drug product, container, and closure are first subjected to sterilization methods separately, as appropriate, and then brought together.

The term “storage-stable,” as used herein, refers to a pharmaceutical composition in which at least about 95% to 99.5% of the active ingredient remains in an undegraded state after storage of the pharmaceutical composition at specified temperature and humidity for a specified time, for example, for 12 months at 25° C. and 60% relative humidity.

The term “substantially free of antimicrobial preservatives” is understood by one of ordinary skill in the art to described a pharmaceutical composition that comprises less than 1% w/w antimicrobial preservatives.

The term “supine,” as used herein, refers to a patient who is lying face up.

The term “t_{1/2}” or “half-life,” as used herein, refers to the amount of time required for half of a drug to be eliminated from the body or the time required for a drug concentration to decline by half.

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

The term “tomography,” as used herein, refers to a process of imaging by sections. The images may be looked at individually, as a series of two-dimensional slices or together, as a computer-generated three-dimensional representation.

The term “pharmaceutically acceptable,” as used herein, refers to a component of a pharmaceutical composition that it compatible with the other ingredients of the formulation and not overly deleterious to the recipient thereof.

The term “substantially free of antimicrobial preservatives” is understood by one of ordinary skill in the art to described a pharmaceutical composition that may comprise less than 1% w/w antimicrobial preservatives.

The term “therapeutically effective amount,” as used herein, refers to the amount of active compound or pharmaceutical agent that elicits the biological or medicinal response in a tissue, system, or individual that is being sought by a researcher, healthcare provider or individual.

The term “Tₘₐₓ,” as used herein, refers to the time from administration of the pharmaceutical compositions described herein to maximum drug plasma concentration.

The term “untrained individual” refers to an individual administering to patient an opioid antagonist using a drug delivery device described herein, wherein the individual is not a healthcare professional and has received no training in the use of the drug delivery device, such as through an overdose education and nasal naloxone distribution (OEND) program.

### Opioid Agonists

Opioid agonists (i.e. opioid receptor agonists or opioids) are a well recognized class of chemical agents that exhibit opium or morphine-like properties. They have been described in detail in the scientific and patent literature. Opioids interact with saturable binding sites in the brain and other tissues, and while they are employed primarily as moderate to strong analgesics, they exhibit many other pharmacological effects including respiratory and/or central nervous system depression.

Provided are co-packaged drug products comprising a therapeutically effective amount of an opioid agonist, and a therapeutically effective amount of an opioid antagonist contained in a drug delivery device selected from: a pre-primed device adapted for nasal delivery of a pharmaceutical composition to a patient, and a hand-held auto-injector adapted for intramuscular or subcutaneous delivery.

In some embodiments, the opioid agonist is selected from: alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof. In some embodiments, the opioid agonist is selected from: alfentanil, and pharmaceutically acceptable salts thereof. In some embodiments, the opioid agonist is selected from: buprenorphine, and pharmaceutically acceptable salts thereof. In some embodiments, the opioid agonist is selected from: butorphanol, and pharmaceutically acceptable salts thereof. In some embodiments, the opioid agonist is selected from: codeine, and pharmaceutically acceptable salts thereof. In some embodiments, the opioid agonist is selected from: diamorphine, and pharmaceutically acceptable salts thereof. In some embodiments, the opioid agonist is selected from: dextromoramide, and pharmaceutically acceptable salts thereof. In some embodiments, the opioid agonist is selected from: dezocine, and pharmaceutically acceptable salts thereof. In some embodiments, the opioid agonist is selected from: dihydrocodeine, and pharmaceutically acceptable salts thereof. In some embodiments, the opioid agonist is selected from: fentanyl, and pharmaceutically acceptable salts thereof. In some embodiments, the opioid agonist is selected from: hydrocodone, and pharmaceutically acceptable salts thereof. In some embodiments, the opioid agonist is selected from: hydromorphone, and pharmaceutically acceptable salts thereof. In some embodiments, the opioid agonist is selected from: levorphanol, and pharmaceutically acceptable salts thereof. In some embodiments, the opioid agonist is selected from: meperidine, and pharmaceutically acceptable salts thereof. In some embodiments, the opioid agonist is selected from: meptazinol, and pharmaceutically acceptable salts thereof. In some embodiments, the opioid agonist is selected from: methadone, and pharmaceutically acceptable salts thereof. In some embodiments, the opioid agonist is selected from: morphine, and pharmaceutically acceptable salts thereof. In some embodiments, the opioid agonist is selected from: nalbuphine, and pharmaceutically acceptable salts thereof. In some embodiments, the opioid agonist is selected from: nalorphine, and pharmaceutically acceptable salts thereof. In some embodiments, the opioid agonist is opium. In some embodiments, the opioid agonist is selected from: oxycodone, and pharmaceutically acceptable salts thereof. In some embodiments, the opioid agonist is selected from: oxymorphone, and pharmaceutically acceptable salts thereof. In some embodiments, the opioid agonist is selected from: selected from: pentazocine, and pharmaceutically acceptable salts thereof. In some embodiments, the opioid agonist is selected from: propoxyphene, and pharmaceutically acceptable salts thereof. In some embodiments, the opioid agonist is selected from: remifentanyl, and pharmaceutically acceptable salts thereof. In some embodiments, the opioid agonist is selected from: sufentanyl, and pharmaceutically acceptable salts thereof. In some embodiments, the opioid agonist is selected from: tapentadol, and pharmaceutically acceptable salts thereof. In some embodiments, the opioid agonist is selected from: tramadol, and pharmaceutically acceptable salts thereof. In some embodiments, the opioid agonist is selected from codeine, morphine, methadone, fentanyl, oxycodone HCl, hydrocodone bitartrate, hydromorphone, oxymorphone, meperidine, propoxyphene, opium, heroin, and certain narcotic-antagonist analgesics, such as, nalbuphine, pentazocine and butorphanol. In some embodiments, the opioid agonist is selected from tapentadol and tramadol.

Tamper-proof and tamper-resistant formulating technologies have been developed for safer delivery of opioid agonists, but such formulations are still abused resulting in opioid overdose. One such technology (Abuse Deterrent Prolonged Release Erosion Matrix (ADPREM); Egalet) utilizes a water-degradable polymer matrix technology that erodes from the surface at a constant rate. The matrix consists of one or more plasticizing polymers that cannot be crushed or melted. Another such technology (Abuse Resistant Technology (ART); Elite Laboratories) utilizes a proprietary coating technology consisting of various polymers that can sequester an opioid antagonist (naltrexone) in fragile micropellets that are indistinguishable from the pellets containing the opioid. The formulation is designed to release sequestered antagonist only if the dosage is crushed or otherwise damaged for extraction. Oral dosage forms are prepared by coating powders, crystals, granules, or pellets with various polymers to impart different characteristics. The formulations can release the active drug in both immediate and sustained release form. Chronodelivery formulations using this technology can effectively delay drug absorption for up to five hours. Aversion (Acura Pharmaceuticals) utilizes certain proprietary combinations of functional excipients (e.g., gelling agents) and active ingredients intended to discourage the most common methods of prescription drug misuse and abuse. Ingredients may include nasal irritants (e.g., capsaicin) and aversive agents (e.g., niacin). In some embodiments, the opioid agonist is in a tamper-proof formulation. In some embodiments, the opioid agonist is in a tamper-resistant formulation. In some embodiments, the opioid agonist is selected from Acurox® Oxycodone DETERx®, Egalet hydrocodone, Egalet morphine, Egalet oxycodone, Exalgo®, Opana®, and Remoxy®.

### Opioid Antagonists

Opioid receptor antagonists (i.e. opioid antagonists) are a well recognized class of chemical agents. They have been described in detail in the scientific and patent literature. Pure opioid antagonists, such as naloxone, are agents which specifically reverse the effects of opioid agonists but have no opioid agonist activity.

Naloxone is commercially available as a hydrochloride salt. Naloxone hydrochloride (17-allyl-4,5a-epoxy-3,14-dihydroxymorphinan-6-one hydrochloride), a narcotic antagonist, is a synthetic congener of oxymorphone. In structure it differs from oxymorphone in that the methyl group on the nitrogen atom is replaced by an allyl group. Naloxone hydrochloride is an essentially pure narcotic antagonist, i.e., it does not possess the “agonistic” or morphine-like properties characteristic of other narcotic antagonists; naloxone does not produce respiratory depression, psychotomimetic effects or pupillary constriction. In the absence of narcotics or agonistic effects of other narcotic antagonists it exhibits essentially no pharmacologic activity. Naloxone has not been shown to produce tolerance or to cause physical or psychological dependence. In the presence of physical dependence on narcotics naloxone will produce withdrawal symptoms.

While the mechanism of action of naloxone is not fully understood, the preponderance of evidence suggests that naloxone antagonizes the opioid effects by competing for the same receptor sites. When naloxone hydrochloride is administered intravenously the onset of action is generally apparent within two minutes; the onset of action is only slightly less rapid when it is administered subcutaneously or intramuscularly. The duration of action is dependent upon the dose and route of administration of naloxone hydrochloride. Intramuscular administration produces a more prolonged effect than intravenous administration. The requirement for repeat doses of naloxone, however, will also be dependent upon the amount, type and route of administration of the narcotic being antagonized. Following parenteral administration naloxone hydrochloride is rapidly distributed in the body. It is metabolized in the liver, primarily by glucuronide conjugation, and excreted in urine. In one study the serum half-life in adults ranged from 30 to 81 minutes (mean 64±12 minutes). In a neonatal study the mean plasma half-life was observed to be 3.1±0.5 hours.

Provided are co-packaged drug products comprising a therapeutically effective amount of an opioid agonist, and a therapeutically effective amount of an opioid antagonist contained in a drug delivery device selected from: a pre-primed device adapted for nasal delivery of a pharmaceutical composition to a patient, and a hand-held auto-injector adapted for intramuscular or subcutaneous delivery.

Also provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a pre-primed device adapted for nasal delivery of a pharmaceutical composition to a patient; and wherein the therapeutically effective amount, is equivalent to about 4 mg to about 12 mg of naloxone hydrochloride.

Also provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a pre-primed device adapted for nasal delivery of a pharmaceutical composition to a patient; and wherein the therapeutically effective amount, is equivalent to about 0.4 mg to about 12 mg of naloxone hydrochloride.

Also provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a pre-primed device adapted for nasal delivery of a pharmaceutical composition to a patient; and wherein the therapeutically effective amount is equivalent to about 3.4 mg to about 12 mg of naloxone hydrochloride. In some embodiments, the therapeutically effective amount is equivalent to about 2 mg to about 24 mg of naloxone hydrochloride. In some embodiments, the therapeutically effective amount is equivalent to about 3 mg to about 18 mg of naloxone hydrochloride. In some embodiments, the therapeutically effective amount is equivalent to about 5 mg to about 11 mg of naloxone hydrochloride. In some embodiments, the therapeutically effective amount is equivalent to about 6 mg to about 10 mg of naloxone hydrochloride. In some embodiments, the therapeutically effective amount is equivalent to about 0.4 mg to about 12 mg of naloxone hydrochloride. In some embodiments, the therapeutically effective amount is equivalent to about 7 mg to about 9 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the therapeutically effective amount is equivalent to about 5 mg of naloxone hydrochloride. In some embodiments, the therapeutically effective amount is equivalent to about 6 mg of naloxone hydrochloride. In some embodiments, the therapeutically effective amount is equivalent to about 7 mg of naloxone hydrochloride. In some embodiments, the therapeutically effective amount is equivalent to about 8 mg of naloxone hydrochloride. In some embodiments, the therapeutically effective amount is equivalent to about 9 mg of naloxone hydrochloride. In some embodiments, the therapeutically effective amount is equivalent to about 10 mg of naloxone hydrochloride. In some embodiments, the therapeutically effective amount is equivalent to about 11 mg of naloxone hydrochloride. In some embodiments, the therapeutically effective amount is equivalent to about 12 mg of naloxone hydrochloride. In some embodiments, the opioid antagonist is the only pharmaceutically active compound in pharmaceutical composition. In some embodiments, the opioid antagonist is naloxone hydrochloride. In some embodiments, the opioid antagonist is anhydrous naloxone hydrochloride. In some embodiments, the opioid antagonist is naloxone hydrochloride dihydrate.

Also provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a hand-held auto-injector adapted for intramuscular or subcutaneous delivery of a pharmaceutical composition to a patient; and wherein the therapeutically effective amount of the opioid antagonist is equivalent to about 0.02 mg to about 2 mg of naloxone hydrochloride. In some embodiments, the therapeutically effective amount is equivalent to about 0.1 mg to about 1 mg of naloxone hydrochloride. In some embodiments, the therapeutically effective amount is equivalent to about 0.2 mg to about 0.6 mg of naloxone hydrochloride. In some embodiments, the therapeutically effective amount is equivalent to about 0.4 mg of naloxone hydrochloride. In some embodiments, the therapeutically effective amount is equivalent to about 0.4 mg of naloxone hydrochloride. In some embodiments, the opioid antagonist is the only pharmaceutically active compound in pharmaceutical composition. In some embodiments, the opioid antagonist is naloxone hydrochloride. In some embodiments, the opioid antagonist is anhydrous naloxone hydrochloride. In some embodiments, the opioid antagonist is naloxone hydrochloride dihydrate.

While many of the embodiments of the pharmaceutical compositions described herein will be described and exemplified with naloxone, other opioid antagonists can be adapted for nasal delivery based on the teachings of the specification. In fact, it should be readily apparent to one of ordinary skill in the art from the teachings herein that the drug delivery devices and pharmaceutical compositions described herein may be suitable for other opioid antagonists. The opioid receptor antagonists described herein include μ-opioid antagonists and δ-opioid receptor antagonists. Examples of useful opioid receptor antagonists include naloxone, naltrexone, methylnaltrexone, nalmefene, Other useful opioid receptor antagonists are known (e.g., Kreek et al., U.S. Pat. No. 4,987,136)

Also provided are co-packaged drug products comprising a therapeutically effective amount of an opioid agonist, and a therapeutically effective amount of an opioid antagonist; wherein the opioid antagonist is contained in a pre-primed device adapted for nasal delivery of a pharmaceutical composition to a patient; and wherein the therapeutically effective amount, is equivalent to about 0.4 mg to about 12 mg of naloxone hydrochloride. In some embodiments, the therapeutically effective amount is equivalent to about 4 mg to about 12 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the opioid antagonist is selected from naltrexone, methylnaltrexone, and nalmefene, and pharmaceutically acceptable salts thereof. In some embodiments, the opioid antagonist is naltrexone hydrochloride. In some embodiments, the opioid antagonist is the only pharmaceutically active compound in pharmaceutical composition.

Also provided are co-packaged drug products comprising a therapeutically effective amount of an opioid agonist, and a therapeutically effective amount of an opioid antagonist; wherein the opioid antagonist is contained in a hand-held auto-injector adapted for intramuscular or subcutaneous delivery of a pharmaceutical composition to a patient; and wherein the therapeutically effective amount of the opioid antagonist is equivalent to about 0.02 mg to about 2 mg of naloxone hydrochloride. In some embodiments, the therapeutically effective amount is equivalent to about 0.4 mg of naloxone hydrochloride. In some embodiments, the opioid antagonist is selected from naltrexone, methylnaltrexone, and nalmefene, and pharmaceutically acceptable salts thereof. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the opioid antagonist is the only pharmaceutically active compound in pharmaceutical composition.

### Nasal Drug Delivery Devices

Provided are co-packaged drug products comprising a pre-primed device adapted for nasal delivery of a pharmaceutical composition described herein. Nasal delivery is considered an attractive route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions and emulsions can also be delivered. In traditional spray pump systems, antimicrobial preservatives are typically required to maintain microbiological stability in liquid formulations.

Some EMS programs have developed a system using existing technologies of an approved drug and an existing medical device to administer naloxone intranasally, albeit in a non-FDA approved manner. This has been accomplished by using the injectable formulation (1 mg/mL) and administering 1 mL per nostril via a marketed nasal atomizer/nebulizer device. The system combines an FDA-approved naloxone injection product (with a Luer fitted tip, no needles) with a marketed, medical device called the Mucosal Atomization Device (MAD™ Nasal, Wolfe Tory Medical, Inc.). This initiative is consistent with the U.S. Needlestick Safety and Prevention Act (Public Law 106-430). The EMS programs recognize limitations of this system, one limitation being that it is not assembled and ready-to-use. Although this administration mode appears to be effective in reversing narcosis, the formulation is not concentrated for retention in the nasal cavity. The 1 mL delivery volume per nostril is larger than that generally utilized for intranasal drug administration. Therefore, there is loss of drug from the nasal cavity, due either to drainage into the nasopharynx or externally from the nasal cavity. The devices described herein are improved ready-to-use products specifically optimized, concentrated, and formulated for nasal delivery.

Metered spray pumps have dominated the nasal drug delivery market since they were introduced. The pumps typically deliver 100 μL (25-200 μL) per spray, and they offer high reproducibility of the emitted dose and plume geometry in in vitro tests. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume (www.aptar.com and www.rexam.-com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a headdown application is recommended. Another method used for avoiding preservatives is that the air that replaces the emitted liquid is filtered through an aseptic air filter. In addition, some systems have a ball valve at the tip to prevent contamination of the liquid inside the applicator tip (www.aptar.com). More recently, pumps have been designed with side-actuation and introduced for delivery of fluticasone furoate for the indication of seasonal and perennial allergic rhinitis. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (www.rexam.com and www.aptar.com).

Metered-dose spray pumps require priming and some degree of overfill to maintain dose conformity for the labeled number of doses. They are well suited for drugs to be administered daily over a prolonged duration, but due to the priming procedure and limited control of dosing, they are less suited for drugs with a narrow therapeutic window. For expensive drugs and vaccines intended for single administration or sporadic use and where tight control of the dose and formulation is of particular importance, single-dose or bi-dose spray devices are preferred (www.aptar.com). A simple variant of a single-dose spray device (MAD™) is offered by LMA (LMA, Salt Lake City, Utah, USA; www.lmana. com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, NC, USA; www.bdpharma.com) is used to deliver the influenza vaccine FluMist (www.flumist.com), approved for both adults and children in the US market. A similar device for two doses was marketed by a Swiss company for delivery of another influenza vaccine a decade ago. The single- and bi-dose devices mentioned above consist of a reservoir, a piston, and a swirl chamber (see, e.g., the UDS UnitDose and BDS BiDose devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex (www.gsk.com) and Zomig (www.az.com; Pfeiffer/Aptar single-dose device) and the marketed influenza vaccine Flu-Mist (www.flumist.com; Becton Dickinson single-dose spray device) are delivered with this type of device.

With sterile filling, the use of preservatives is not required, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex (sumatriptan) and Zomig (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

Provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a pre-primed device adapted for nasal delivery of a pharmaceutical composition to a patient; and wherein the therapeutically effective amount, is equivalent to about 0.4 mg to about 12 mg of naloxone hydrochloride. In some embodiments, the therapeutically effective amount is equivalent to about 4 mg to about 12 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the therapeutically effective amount of the opioid antagonist is equivalent to about 4 mg to about 12 mg of naloxone hydrochloride. In some embodiments, the opioid antagonist is equivalent to about 6 mg to about 10 mg of naloxone hydrochloride. In some embodiments, the opioid antagonist is equivalent to about 8 mg of naloxone hydrochloride. In some embodiments, the opioid antagonist is the only pharmaceutically active compound in the delivery device. In some embodiments, the opioid antagonist is naloxone hydrochloride.

In some embodiments, the drug delivery device is a single-dose device, wherein the pharmaceutical composition is present in one reservoir, and wherein the therapeutically effective amount of the opioid antagonist is delivered essentially by one actuation of the drug delivery device into one nostril of the patient. In some embodiments, the volume of the pharmaceutical composition in the reservoir is not more than about 140 μL. In some embodiments, the volume of the pharmaceutical composition in the reservoir is about 100 μL to about 140 μL. In some embodiments, the volume of the pharmaceutical composition in the reservoir is about 125 μL. In some embodiments, about 80% to about 90% of the pharmaceutical composition in the reservoir is delivered by one actuation. In some embodiments, about 85% of the pharmaceutical composition in the reservoir is delivered by one actuation. In some embodiments, about 100 μL of the pharmaceutical composition in the reservoir is delivered to the patient in one actuation.

In some embodiments, the drug delivery device is a bi-dose device, wherein a first volume of the pharmaceutical composition is present in a first reservoir and a second volume of the pharmaceutical composition is present in a second reservoir, and wherein the therapeutically effective amount is delivered essentially by a first actuation of the drug delivery device into a first nostril of the patient and a second actuation of the drug delivery device into a second nostril of the patient. In some embodiments, the first volume and the second volume combined is equal to not more than about 380 μL. In some embodiments, the first volume of the pharmaceutical composition is not more than about 190 μL. In some embodiments, the first volume of the pharmaceutical composition is about 100 μL to about 190 μL. In some embodiments, the first volume of the pharmaceutical composition is about 125 μL. In some embodiments, about 80% to about 90% of the first volume of the pharmaceutical composition is delivered by the first actuation. In some embodiments, about 85% of the first volume of the pharmaceutical composition is delivered by the first actuation. In some embodiments, about 100 μL of the first volume of the pharmaceutical composition is delivered by the first actuation. In some embodiments, the second volume of the pharmaceutical composition is not more than about 190 μL. In some embodiments, the second volume of the pharmaceutical composition is about 100 μL to about 190 μL. In some embodiments, the second volume of the pharmaceutical composition is about 125 μL. In some embodiments, about 80% to about 90% of the second volume of the pharmaceutical composition is delivered by the second actuation. In some embodiments, about 85% of the second volume of the pharmaceutical composition is delivered by the second actuation. In some embodiments, about 100 μL of the second volume of the pharmaceutical composition is delivered by the second actuation.

In some embodiments, the drug delivery device is actuatable with one hand. In some embodiments, the delivery time is less than about 25 seconds. In some embodiments, the delivery time is less than about 20 seconds. In some embodiments, the 90% confidence interval for dose delivered per actuation is ± about 2%. In some embodiments, the 95% confidence interval for dose delivered per actuation is ± about 2.5%. In some embodiments, the 99% confidence interval for dose delivered per actuation is ± about 3%. In some embodiments, upon nasal delivery of the pharmaceutical composition to the patient using the drug delivery device, less than about 20% of the pharmaceutical composition leaves the nasal cavity via drainage into the nasopharynx or externally. In some embodiments, upon nasal delivery of the pharmaceutical composition to the patient using the drug delivery device, less than about 10% of the pharmaceutical composition leaves the nasal cavity via drainage into the nasopharynx or externally. In some embodiments, upon nasal delivery of the pharmaceutical composition to the patient using the drug delivery device, less than about 5% of the pharmaceutical composition leaves the nasal cavity via drainage into the nasopharynx or externally.

In some embodiments, the plasma concentration versus time curve of the opioid antagonist in the patient has a Tₘₐₓ of less than 20 minutes. In some embodiments, the plasma concentration versus time curve of the opioid antagonist in the patient has a Tₘₐₓ of less than 19 minutes. In some embodiments, the plasma concentration versus time curve of the opioid antagonist in the patient has a Tₘₐₓ of about 18.5 minutes.

### Auto-Injector Drug Delivery Devices

Auto-injector drug delivery devices are well known in the scientific and patent literature. An auto-injector is a device for enabling an individual to administer a dosage of a liquid medicament. An advantage of auto-injectors is that they contain a measured dosage of a liquid medicament in a sealed sterile condition capable of storage in such condition for an extensive period of non-use, during which period immediate injection of the stored dosage may be accomplished at any time under the most severe emergency conditions. An auto-injector may include a releasable stressed spring assembly. This assembly includes a stressed spring, a releasable mechanism for releasably retaining the spring in a stressed storage position and a releasing mechanism for releasing the releasable mechanism in response to a predetermined actuating procedure. Auto-injectors are particularly suited for use under emergency conditions. For example, many tens of millions of such auto-injectors have been manufactured and sold containing nerve gas antidotes for use under emergency chemical warfare conditions. Typical units which have been utilized for this purpose are disclosed in U.S. Pat. Nos. 2,832,339, 3,882,863, and 4,031,893. In addition, units of this type have been proposed for use in administering antiarrhythmic medicaments under emergency conditions relating to heart attack medical situations. Such use has been in conjunction with portable monitors as is evident from the disclosure contained in U.S. Pat. Nos. 3,910,260 and 4,004,577. It has also been proposed to provide other medicaments useful in treating heart attack symptoms such as clot selective thrombolytic agents (e.g. tissue plasminogen activator) and related medicaments. See, for example, U.S. Pat. Nos. 4,689,042, 4,755,169, and 4,795,433. Auto-injectors have been marketed in recent years containing a dosage of epinephrine as an antidote for counteracting severe allergic reactions, as for example, to bee stings and the like. In all of these instances, the emergency use aspect of the auto-injectors is of primary significance.

Evzio is an opioid antagonist indicated for the emergency treatment of known or suspected opioid overdose, as manifested by respiratory and/or central nervous system depression. Evzio is injected into the muscle (intramuscular) or under the skin (subcutaneous). In one pharmacokinetic study of 30 patients, a single Evzio injection provided equivalent naloxone compared to a single dose of naloxone injection using a standard syringe. Once turned on, the device provides verbal instruction to the user describing how to deliver the medication, similar to automated defibrillators. If the Evzio electronic voice instruction system does not operate properly, Evzio will still deliver the intended dose of naloxone hydrochloride when used according to the printed instructions on its label. Upon actuation, Evzio automatically inserts the needle intramuscularly or subcutaneously, delivers 0.4 mg naloxone hydrochloride injection, and retracts the needle fully into its housing. Post injection, the black base locks in place, a red indicator appears in the viewing window, and electronic visual and audible instructions signal that Evzio has delivered the intended dose of naloxone hydrochloride and instructs the user to seek emergency medical attention. The requirement for repeat doses of Evzio depends upon the amount, type, and route of administration of the opioid being antagonized. If the desired response is not obtained after 2 or 3 minutes, another Evzio dose may be administered. If there is still no response and additional doses are available, additional Evzio doses may be administered every 2 to 3 minutes until emergency medical assistance arrives. Additional supportive and/or resuscitative measures may be helpful while awaiting emergency medical assistance. Reversal of respiratory depression by partial agonists or mixed agonist/antagonists, such as buprenorphine and pentazocine, may be incomplete or require higher doses of naloxone. In pediatric patients under the age of one, the caregiver should pinch the thigh muscle while administering Evzio.

Provided are co-packaged drug products comprising a hand-held auto-injector adapted for intramuscular or subcutaneous delivery of a pharmaceutical composition described herein. In some embodiments, the drug delivery device is a hand-held auto-injector adapted for intramuscular or subcutaneous delivery of a pharmaceutical composition to a patient, and the therapeutically effective amount of the opioid antagonist is equivalent to about 0.02 mg to about 2 mg of naloxone hydrochloride. In some embodiments, the drug delivery device is a hand-held auto-injector, and the therapeutically effective amount of the opioid antagonist is equivalent to about 0.4 mg of naloxone hydrochloride. In some embodiments, the opioid antagonist is the only pharmaceutically active compound in the delivery device. In some embodiments, the opioid antagonist is naloxone hydrochloride.

### Pharmaceutical Compositions

Provided are co-packaged drug products comprising a therapeutically effective amount of an opioid agonist, and a therapeutically effective amount of an opioid antagonist selected from: naloxone and pharmaceutically acceptable salts thereof; wherein said opioid antagonist is contained in a drug delivery device selected from: a pre-primed device adapted for nasal delivery of a pharmaceutical composition to a patient, and a hand-held auto-injector adapted for intramuscular or subcutaneous delivery of a pharmaceutical composition to a patient.

In some embodiments, the pharmaceutical composition comprises a solution of naloxone hydrochloride. In some embodiments, the pharmaceutical composition further comprises one or more excipients selected from water and NaCl. In some embodiments, the pharmaceutical composition is substantially free of antimicrobial preservatives. In some embodiments, the drug delivery device is filled with the pharmaceutical composition using sterile filling. In some embodiments, the pharmaceutical composition is storage-stable for about twelve months at about 25° C. and about 60% relative humidity.

In some embodiments the pharmaceutical compositions comprise an opioid antagonist and a pharmaceutically acceptable carrier. The carrier(s) must be “acceptable” in the sense of being compatible with the other ingredients of the formulation and not overly deleterious to the recipient thereof. Some embodiments of the present invention include a method of producing a pharmaceutical composition comprising admixing at least one opioid antagonist and a pharmaceutically acceptable carrier. Pharmaceutical compositions are applied directly to the nasal cavity using the drug delivery devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof; and a suspending agent such as microcrystalline cellulose, carboxymethylcellulose sodium NF, polyacrylic acid, magnesium aluminum silicate, xanthan gum, and the like, and mixtures thereof.

The opioid antagonists described herein can be formulated into pharmaceutical compositions using techniques well known to those in the art. Suitable pharmaceutically acceptable carriers, outside those mentioned herein, are known in the art; for example, see Remington: The Science and Practice of Pharmacy, 21st ed., Lippincott Williams & Wilkins, Philadelphia, Pa. (2005).

The opioid antagonists described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. The opioid antagonists described herein may form solvates with standard low molecular weight solvents using methods known to the skilled artisan.

Provided are co-packaged drug products comprising a therapeutically effective amount of an opioid agonist, and a therapeutically effective amount of an opioid antagonist selected from: naloxone and pharmaceutically acceptable salts thereof; wherein said opioid antagonist is contained in a pre-primed device adapted for nasal delivery of a pharmaceutical composition to a patient; and wherein the therapeutically effective amount, is equivalent to about 0.4 mg to about 12 mg of naloxone hydrochloride. In some embodiments, the therapeutically effective amount is equivalent to about 4 mg to about 12 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the pharmaceutical composition comprises a solution of naloxone hydrochloride. In some embodiments, the pharmaceutical composition further comprises one or more excipients selected from water and NaCl. In some embodiments, the pharmaceutical composition is substantially free of antimicrobial preservatives. In some embodiments, the drug delivery device is substantially free of benzalkonium chloride, methylparaben, sodium benzoate, benzoic acid, phenyl ethyl alcohol. In some embodiments, the drug delivery device is filled with the pharmaceutical composition in a sterile environment. In some embodiments, the pharmaceutical composition is storage-stable for about twelve months at about 25° C. In some embodiments, the pharmaceutical composition comprises less than 0.1% w/w antimicrobial preservatives. In some embodiments, the pharmaceutical composition comprises less than 0.01% w/w antimicrobial preservatives. In some embodiments, the pharmaceutical composition comprises less than 0.001% w/w antimicrobial preservatives.

Provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a hand-held auto-injector adapted for intramuscular or subcutaneous delivery of a pharmaceutical composition to a patient; and wherein the therapeutically effective amount of the opioid antagonist is equivalent to about 0.02 mg to about 2 mg of naloxone hydrochloride. In some embodiments, the therapeutically effective amount is equivalent to about 0.1 mg to about 1 mg of naloxone hydrochloride. In some embodiments, the therapeutically effective amount is equivalent to about 0.2 mg to about 0.6 mg of naloxone hydrochloride. In some embodiments, the therapeutically effective amount is equivalent to about 0.4 mg of naloxone hydrochloride. In some embodiments, the therapeutically effective amount is equivalent to about 0.4 mg of naloxone hydrochloride. In some embodiments, the pharmaceutical composition further comprises one or more inactive ingredients selected from: sodium chloride, hydrochloric acid, and water. In some embodiments, the pH range of the pharmaceutical composition is 3.0 to 4.5.

In some embodiments, the opioid antagonist is the only pharmaceutically active compound in pharmaceutical composition. In some embodiments, the opioid antagonist is naloxone hydrochloride. In some embodiments, the opioid antagonist is anhydrous naloxone hydrochloride. In some embodiments, the pharmaceutical composition comprises a solution of naloxone hydrochloride. In some embodiments, the opioid antagonist is selected from naltrexone, methylnaltrexone, and nalmefene, and pharmaceutically acceptable salts thereof. In some embodiments, the opioid antagonist is naltrexone hydrochloride. In some embodiments, the opioid antagonist is methylnaltrexone bromide. In some embodiments, the opioid antagonist is nalmefene hydrochloride.

Also provided are co-packaged drug products comprising a therapeutically effective amount of an opioid agonist, and a device described herein for “combination-therapy” comprising pharmaceutical compositions comprising at least one opioid antagonist described herein, together with at least one known pharmaceutical agent and a pharmaceutically acceptable carrier. In some embodiments, the pharmaceutical composition comprises a short-acting opioid antagonist and a long-acting opioid antagonist. In some embodiments, the pharmaceutical composition comprises naloxone and naltrexone. In some embodiments, the pharmaceutical composition comprises naloxone and methylnaltrexone. In some embodiments, the pharmaceutical composition comprises naloxone and nalmefene.

### Indications

Naloxone prevents or reverses the effects of opioids including respiratory depression, sedation and hypotension. Also, it can reverse the psychotomimetic and dysphoric effects of agonist-antagonists such as pentazocine. Naloxone causes abrupt reversal of narcotic depression which may result in nausea, vomiting, sweating, tachycardia, increased blood pressure, tremulousness, seizures and cardiac arrest, however, there is no clinical experience with naloxone hydrochloride overdosage in humans. In the mouse and rat the intravenous LD50 is 150±5 mg/kg and 109±4 mg/kg respectively. In acute subcutaneous toxicity studies in newborn rats the LD50 (95% CL) is 260 (228-296) mg/kg. Subcutaneous injection of 100 mg/kg/day in rats for 3 weeks produced only transient salivation and partial ptosis following injection: no toxic effects were seen at 10 mg/kg/day for 3 weeks.

Naloxone hydrochloride injection is indicated for the complete or partial reversal of narcotic depression, including respiratory depression, induced by opioids selected from: natural and synthetic narcotics, propoxyphene, methadone, and certain narcotic-antagonist analgesics: nalbuphine, pentazocine and butorphanol. Naloxone hydrochloride is also indicated for the diagnosis of suspected acute opioid overdosage. For the treatment of known or suspected narcotic overdose in adults an initial dose of 0.4 mg to 2 mg of naloxone hydrochloride intravenously is indicated. If the desired degree of counteraction and improvement in respiratory functions is not obtained, administration may be repeated at 2 to 3 minute intervals. If no response is observed after 10 mg of naloxone hydrochloride have been administered, the diagnosis of narcotic-induced or partial narcotic-induced toxicity should be questioned. The usual initial dose in children is 0.01 mg/kg body weight given IV. If this dose does not result in the desired degree of clinical improvement, a subsequent dose of 0.1 mg/kg body weight may be administered. When using naloxone hydrochloride injection in neonates a product containing 0.02 mg/mL should be used.

It has also been reported that naloxone hydrochloride is an effective agent for the reversal of the cardiovascular and respiratory depression associated with narcotic and possibly some non-narcotic overdoses. The authors stated that due to naloxone's pharmacokinetic profile, a continuous infusion protocol is recommended when prolonged narcotic antagonist effects are required. (Handal et al., Ann Emerg Med. 1983 July; 12(7):438-45).

Provided are methods of lowering opioid overdose risk comprising the steps of: co-packaging a therapeutically effective amount of an opioid agonist, and a therapeutically effective amount of an opioid antagonist selected from: naloxone and pharmaceutically acceptable salts thereof, to form a co-packaged drug product; prescribing the co-packaged drug product to a first individual; and dispensing the co-packaged drug product to the first individual or a second individual; wherein the opioid antagonist is contained in a drug delivery device selected from: a pre-primed device adapted for nasal delivery of a pharmaceutical composition to a patient, and a hand-held auto-injector adapted for intramuscular or subcutaneous delivery of a pharmaceutical composition to a patient.

In some embodiments, the method further comprises counseling the first individual or the second individual on the use of the co-packaged drug product.

In some embodiments, the opioid antagonist is for use in the treatment of an opioid overdose symptom. In some embodiments, the opioid overdose symptom is selected from: respiratory depression, altered level consciousness, miotic pupils, cardiovascular depression, hypoxemia, acute lung injury, aspiration pneumonia, sedation, and hypotension. In some embodiments, the opioid antagonist is for use in the emergency treatment of known or suspected opioid overdose, as manifested by one or more symptoms selected from: respiratory depression and central nervous system depression. In some embodiments, the opioid antagonist is for use in the emergency treatment of known or suspected opioid overdose characterized by one or more symptoms selected from: decreased breathing rate, decreased heart rate, and loss of consciousness. In some embodiments, the symptom is respiratory depression. In some embodiments, the opioid antagonist is for use in the complete or partial reversal of narcotic depression, including respiratory depression, induced by opioids selected from: natural and synthetic narcotics, propoxyphene, methadone, nalbuphine, pentazocine and butorphanol.

In some embodiments, the opioid overdose symptom is respiratory depression induced by opioids. In some embodiments, the respiratory depression is induced by opioids selected from: natural and synthetic narcotics, propoxyphene, methadone, nalbuphine, pentazocine and butorphanol. In some embodiments, the respiratory depression is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol. In some embodiments, the respiratory depression is caused by the illicit use of opioids or by an accidental misuse of opioids during medical opioid therapy. In some embodiments, the symptom is caused by misuse of the opioid agonist.

In some embodiments, the patient is in a lying position. In some embodiments, the patient is in a supine position. In some embodiments, the patient is in a prone position. In some embodiments, the patient is not breathing. In some embodiments, the patient is an opioid overdose patient. In some embodiments, the therapeutically effective amount of an opioid antagonist is delivered by an untrained individual.

In some embodiments, the patient is free from respiratory depression for at least about 1 hour following treatment consisting essentially of delivery of the therapeutically effective amount of the opioid antagonist. In some embodiments, the patient is free from respiratory depression for at least about 2 hours following treatment consisting essentially of delivery of the therapeutically effective amount of the opioid antagonist. In some embodiments, the patient is free from respiratory depression for at least about 3 hours following treatment consisting essentially of delivery of the therapeutically effective amount of the opioid antagonist. In some embodiments, the patient is free from respiratory depression for at least about 4 hours following treatment consisting essentially of delivery of the therapeutically effective amount of the opioid antagonist. In some embodiments, the patient is free from respiratory depression for at least about 5 hours following treatment consisting essentially of delivery of the therapeutically effective amount of the opioid antagonist. In some embodiments, the patient is free from respiratory depression for at least about 6 hours following treatment consisting essentially of delivery of the therapeutically effective amount of the opioid antagonist.

### Receptor Occupancy

Provided are co-packaged drug products comprising an opioid receptor agonist, and an opioid receptor antagonist in a delivery device, which provides a high level of brain opioid receptor occupancy as may be determined, for example, by positron emission tomography (PET). PET and single-photon emission computed tomography (SPECT) are noninvasive imaging techniques that can give insight into the relationship between target occupancy and drug efficacy, provided a suitable radioligand is available. Although SPECT has certain advantages (e.g., a long half-life of the radionuclides), the spatial and temporal resolution as well as the labeling possibilities of this technique are limited.

PET involves the administration to a subject of a positron-emitting radionuclide tracer followed by detection of the positron emission (annihilation) events in the body. The radionuclide tracer is typically composed of a targeting molecule having incorporated therein one or more types of positron-emitting radionuclides. Positron-emitting radionuclides include ¹¹C, ¹³N, ¹⁵O, ¹⁸F, ⁵²Fe, ⁶²Cu, ⁶⁴Cu, ⁶⁸Ga, ⁷⁴As, ⁸²Rb, ⁸⁹zr, ¹²²I, and ¹²⁴I. Non-metal radionuclides may be covalently linked to the targeting molecule by reactions well known from the state of art. When the radionuclide is a metallic positron-emitter, it is understood that labeling may require the use of a chelating agent. Such chelating agents are well known from the state of the art.

The positron-emitter labeled compound is administered directly, e.g., IV, or indirectly, e.g., IN, into the subject's vascular system, from where it passes through the blood-brain barrier. Once the tracer has had sufficient time to associate with the target of interest, the individual is placed within in a scanning device comprising ring of scintillation detectors. An emitted positron travels through the individual's tissue for a short (isotope-dependent) distance, until it interacts with an electron. The interaction annihilates both the electron and the positron, producing a pair of photons moving in approximately opposite directions. These are detected when they reach a scintillator in the scanning device. Photons that do not arrive in pairs are ignored. An image is then generated of the part of the individual's brain to which the compound has distributed.

PET studies are useful for comparing nasal delivery of naloxone using the drug delivery devices and at the doses described herein, to typical nasal doses of naloxone (such as 1-2 mg), to delivery of naloxone using other nasal devices (such as the MAD™) and by other routes of administration such IM or IV naloxone or oral naltrexone or nalmefene. Further comparisons may be made between nasal administration in the upright versus the lying or supine positions. Useful measures that may be determined in such studies are the time to onset of action, brain half-life, and the percent receptor binding or occupancy of a patient's opioid receptors, for example, the μ-opioid receptors in the respiratory center in the medulla oblongata.

[¹¹C]Carfentanil (CFN) is a μ-opioid agonist used for in vivo PET studies of μ-opioid receptors. One such study involved healthy male volunteers assigned at enrolment to receive either naltrexone or a novel μ-opioid receptor inverse agonist (GSK1521498) (Rabiner et al., Pharmacological differentiation of opioid receptor antagonists by molecular and functional imaging of target occupancy and food reward-related brain activation in humans. Molecular Psychiatry (2011) 16, 826-835). Each participant underwent up to three [¹¹C]-carfentanil PET scans and two fMRI examinations: one [¹¹C]-carfentanil PET scan and one fMRI scan at baseline (before dosing) and up to two PET scans and one fMRI scan following oral administration of a single dose of GSK1521498 or naltrexone. The administered doses of GSK1521498 or naltrexone were chosen adaptively to optimize the estimation of the dose-occupancy relationship for each drug on the basis of data acquired from the preceding examinations in the study. The administered dose range was 0.4-100 mg for GSK1521498, and 2-50 mg for naltrexone. The maximum doses administered were equal to the maximum tolerated dose of GSK1521498 determined in the first-in-human study and the standard clinical dose of naltrexone used for alcohol dependence. The times and doses of the two post-dose [¹¹C]-carfentanil PET scans were chosen adaptively for each subject to optimize estimation of the relationship between plasma concentration and receptor occupancy. Post-dose [¹¹C]-carfentanil PET scans were acquired at 3-36 h after the administration of GSK1521498 and at 3-88 h after the administration of naltrexone. Post-dose fMRI scans were acquired within 60 min of the first post-dose PET scan. Venous blood samples were collected at regular intervals throughout the scanning sessions. High-performance liquid chromatography/mass spectrometry/mass spectrometry was used to estimate the plasma concentrations of GSK1521498, naltrexone, and the major metabolite of naltrexone, 6-β-naltrexol. Drug plasma concentration at the start of each PET scan was used to model the relationship between drug concentrations and μ-opioid receptor occupancies. Carfentanil (methyl 1-(2-phenylethyl)-4-(phenyl(propanoyl)amino)-4-piperidinecarboxylate 3S, 5S; Advanced Biochemical Compounds, Radeberg, Germany), a potent selective μ-opioid receptor agonist, was labelled with carbon-11 using a modification of a previously described method implemented using a semiautomated Modular Lab Multifunctional Synthetic Module (Eckert & Ziegler, Berlin, Germany). The final product was reformulated in sterile 0.9% saline containing ˜10% ethanol (v/v) and satisfied quality control criteria for specific activity and purity before being injected intravenously as a slow bolus over ˜30 s. PET scanning was conducted in three-dimensional mode using a Siemens Biograph 6 Hi-Rez PET-CT for the naltrexone group and a Siemens Biograph 6 TruePoint PET-CT for the GSK1521498 group (Siemens Healthcare, Erlangen, Germany). A low-dose CT scan was acquired for attenuation correction before the administration of the radiotracer. Dynamic PET data were acquired for 90 min after [¹¹C]-carfentanil injection, binned into 26 frames (durations: 8×15 s, 3×60 s, 5×2 min, 5×5 min and 5×10 min), reconstructed using Fourier re-binning and a two-dimensional-filtered back projection algorithm and then smoothed with a two-dimensional Gaussian filter (5 mm at full width half maximum). Dynamic PET images were registered to each participant's T1-weighted anatomical MRI volume and corrected for head motion using SPM5 software (Welcome Trust Centre for Neuroimaging). Pre-selected regions of interests were defined bilaterally on the T1-weighted anatomical volume using an in-house atlas and applied to the dynamic PET data to generate regional time-activity curves. The [¹¹C]-carfentanil-specific binding was quantified as binding potential relative to the non-displaceable compartment (BP_{ND})

$${BP}_{ND} = \frac{{f}_{ND} {B}_{avail}}{{K}_{D}}$$

where f_{ND} is the free fraction of the radioligand in the brain, K_{D} is the affinity of [¹¹C]-carfentanil, and Bₐᵥₐᵢₗ is the density of the available μ-opioid receptors. Regional [¹¹C]-carfentanil BP_{ND} was estimated using a reference tissue model with the occipital cortex as the reference region. Drug related occupancy of the μ-opioid receptor was quantified as a reduction of [¹¹C]-carfentanil.

$${Occupancy}_{Drug} = \frac{{BP}_{ND}^{Baseline} - {BP}_{ND}^{Drug}}{{BP}_{ND}^{Baseline}}$$

The affinity constant for each drug at the μ-opioid receptor (effective concentration 50 (EGO) was estimated by fitting the plasma concentration measured at the start of the PET scan, C^{P}_{Drug}, to the estimated occupancy:

$${Occupancy}_{Drug} = \frac{{C}_{Drug}^{P}}{{C}_{Drug}^{P} + {EC}_{50}}$$

The use of a sensitive non-tomographic positron detecting system to measure the dose-response curve of naloxone in human brain has also been reported. [¹¹C]Diprenorphine was administered to normal volunteers in tracer amounts and, 30 min later, various bolus doses of naloxone were given (1.5-160 μg/kg) intravenously and change in [¹¹C]diprenorphine binding monitored over the next 30 min. Approximately 13 μg/kg of naloxone (approximately 1 mg in an 80 kg man) was required to produce an estimated 50% receptor occupation, consistent with the clinical dose of naloxone used to reverse opiate overdose (0.4 mg-1.2 mg). Melichar et al., Naloxone displacement at opioid receptor sites measured in vivo in the human brain. Eur J Pharmacol. 2003 Jan. 17; 459(2-3):217-9).

Provided are co-packaged drug products comprising an opioid receptor agonist, and an opioid receptor antagonist in a pre-primed nasal delivery device, comprising a therapeutically effective amount of an opioid antagonist selected from naloxone and pharmaceutically acceptable salts thereof; and wherein the therapeutically effective amount is equivalent to about 0.4 mg to about 12 mg of naloxone hydrochloride. In some embodiments, the therapeutically effective amount is equivalent to about 4 mg to about 12 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, delivery of the therapeutically effective amount to the patient, provides occupancy at Tₘₐₓ of the opioid antagonist at the opioid receptors in the respiratory control center of the patient of greater than about 90%. In some embodiments, delivery of the therapeutically effective amount to the patient, provides occupancy at Tₘₐₓ of the opioid antagonist at the opioid receptors in the respiratory control center of the patient of greater than about 95%. In some embodiments, delivery of the therapeutically effective amount to the patient, provides occupancy at Tₘₐₓ of the opioid antagonist at the opioid receptors in the respiratory control center of the patient of greater than about 99%. In some embodiments, delivery of the therapeutically effective amount to the patient, provides occupancy at Tₘₐₓ of the opioid antagonist at the opioid receptors in the respiratory control center of the patient of about 100%.

### Co-Packaged Drug Products

A co-packaged drug product comprises of two or more separate drug products in their final dosage form, which may be packaged together with appropriate labeling, such as labeling approved by the FDA, to support the combination use. This is in contrast to a fixed dose combination product, in which two or more separate drug ingredients are combined in a single dosage form.

The co-packaged drug products described herein may further comprise protective packaging materials. Protective packaging materials for use in packaging drug products are well known to those of skill in the art. Examples of protective packaging materials suitable for use with pharmaceuticals include, but are not limited to, boxes, cartons, blister packs, bottles, tubes, inhalers, pumps, bags, vials, containers, syringes, auto-injectors, nasal sprays, bottles, and any packaging material suitable for a selected formulation and intended mode of administration and treatment.

Instructions for use may be included in the co-packaged drug product. Instructions typically include a tangible expression describing the technique to be employed in using the components of the co-packaged drug product to effect a desired outcome, such as using the opioid agonist for the management of moderate to severe pain, and using the opioid antagonist to treat opioid overdose or suspected opioid overdose. For example, instructions for use may include instructions to administer the therapeutically effective amount of the opioid antagonist to the opioid-overdose patient or suspected opioid overdose patient using the drug delivery device. In some embodiments the instructions comprise printed matter. Printed matter can be, for example, a book, booklet, brochure, or leaflet. In some embodiments the instructions comprise a pre-recorded media device. In some embodiments, the pre-recorded media device is a pre-recorded audio device that provides verbal instructions to the user. In some embodiments, the pre-recorded media device signals to the user visually and/or audibly that the drug delivery device has delivered the intended dose of the opioid antagonist to the patient. In some embodiments, the pre-recorded media device instructs the user to seek emergency medical attention.

The co-packaged drug products described herein may further comprise instructions for downloading an application to an electronic device, wherein the application enables the use of the opioid antagonist to treat opioid overdose or suspected opioid overdose. In some embodiments, the electronic device is a mobile electronic device. In some embodiments, the electronic device is a hand-held mobile electronic device. Examples of electronic devices suitable for downloading the applications described herein include cellular telephones, smart phones, personal digital assistants, tablet computers, handheld multimedia players with network connectivity, and the like.

Provided are co-packaged drug products comprising a therapeutically effective amount of an opioid agonist, and a therapeutically effective amount of an opioid antagonist selected from: naloxone and pharmaceutically acceptable salts thereof; wherein the opioid antagonist is contained in a drug delivery device selected from: a pre-primed device adapted for nasal delivery of a pharmaceutical composition to a patient, and a hand-held auto-injector adapted for intramuscular or subcutaneous delivery of a pharmaceutical composition to a patient.

In some embodiments, the co-packaged drug product further comprises protective packaging. In some embodiments, the co-packaged drug product further comprises instructions describing use of the opioid agonist. In some embodiments, the co-packaged drug product further comprises instructions describing use of the opioid antagonist. In some embodiments, the opioid antagonist is for treating opioid overdose or suspected opioid overdose. In some embodiments, the co-packaged drug product further comprises printed matter describing the use of the opioid antagonist to treat opioid overdose or suspected opioid overdose. In some embodiments, the co-packaged drug product further comprises a pre-recorded media device describing the use of the opioid antagonist to treat opioid overdose or suspected opioid overdose. In some embodiments, the co-packaged drug product further comprises instructions for downloading an application to a mobile electronic device, wherein the application enables the use of the opioid antagonist to treat opioid overdose or suspected opioid overdose.

In some embodiments, the opioid antagonist is for use in reversing the psychotomimetic and dysphoric effects of agonist-antagonists such as pentazocine. In some embodiments, the opioid antagonist is for use in the diagnosis of suspected acute opioid overdosage. In some embodiments, the opioid antagonist is for use in treating opioid addiction. In some embodiments, the opioid antagonist is for use in treating septic shock. In some embodiments, the opioid antagonist is for use in treating opioid overdose or a symptom thereof, reversing the psychotomimetic and dysphoric effects of agonist-antagonists such as pentazocine, diagnosing suspected acute opioid overdosage, treating opioid addiction, or treating septic shock.

Also provided are embodiments wherein any embodiment described above may be combined with any one or more of these embodiments, provided the combination is not mutually exclusive.

### EXAMPLES

### Example 1

### Pharmacokinetics and Safety of Intranasal Naloxone in Humans (Study 1)

A clinical trial was performed for which the primary objectives were to determine the pharmacokinetics (PK) of 2 intranasal (IN) doses (2 mg and 4 mg) of naloxone compared to a 0.4 mg dose of naloxone administrated intramuscularly (IM) and to identify an appropriate IN dose that could achieve systemic exposure comparable to an approved parenteral dose. The secondary objectives were to determine the safety of IN naloxone, specifically with respect to nasal irritation (erythema, edema, and erosion).

Methodology: This was an inpatient open-label, randomized, 3-period, 3-treatment, 6-sequence, crossover study involving 14 healthy volunteers. Subjects were assigned to one of the 6 sequences with 2 subjects in each sequence (2 sequences had 3 subjects). Each subject received 3 naloxone doses, a single 2 mg IN dose (one spray of 0.1 mL of 10 mg/mL solution in each nostril), a single 4 mg IN dose (2 sprays of 0.1 mL per spray of 10 mg/mL solution in each nostril) and a single 0.4 mg IM dose, in the 3 dosing periods (Table 1). Subjects stayed in the inpatient facility for 11 days to complete the entire study and were discharged on the next day after the last dose. Subjects returned for a final follow-up visit 3-5 days after discharge. After obtaining informed consent, subjects were screened for eligibility to participate in the study including medical history, physical examination, clinical chemistry, coagulation markers, hematology, infectious disease serology, urinalysis, urine drug and alcohol toxicology screen, vital signs and electrocardiogram (ECG). On the day after clinic admission, subjects were administered study drug in randomized order with a 4-day washout period between doses until all three doses were administered. Blood was collected for naloxone PK prior to dosing and approximately 2.5, 5, 10, 15, 20, 30, 45, 60, 120, 180, 240, 300, 360, 480 and 720 min after the start of study drug administration. On days of study drug administration, a 12-lead ECG was performed approximately 60 min prior to dosing and at approximately 60 and 480 min post-dose. Vital signs were measured pre-dose and approximately 30, 60, 120, and 480 min post-dose. On dosing days, the order of assessments was ECG, vital signs, then PK blood collection when scheduled at the same nominal times. ECG and vital signs were collected within the 10-min period before the nominal time of blood collections. At screening, admission, discharge, and follow-up, ECG and vital signs were checked once per day. Vital signs were also checked once on the day after naloxone administration. Clinical laboratory measurements were repeated after the last PK blood draw prior to clinic discharge. AEs were assessed by spontaneous reports by subjects, examination of the nasal mucosa, physical examination, vital signs, ECG, and clinical laboratory parameters.

Main Criteria for Inclusion/Exclusion: Healthy volunteer adults with a body mass index (BMI) of 18-30 kg/m².

Investigational Product, Dose and Mode of Administration: Naloxone given IN was at a dose of 2 mg (1 squirt in each nostril delivered 0.1 mL of 10 mg/mL naloxone) and 4 mg (2 squirts in each nostril delivered 0.2 mL/nostril at 10 mg/mL naloxone, using two devices). IN naloxone was administered using a Pfeiffer (Aptar) BiDose liquid device with the subject in a fully supine position.

Duration of Treatment: Each IN and IM dose was administered once in each subject in random sequence.

Reference Therapy, Dose and Mode of Administration: Naloxone was given IM at a dose of 0.4 mg in 1.0 mL with a 23-g needle as a single injection in the gluteus maximus muscle.

PK Evaluation: Blood was collected in sodium heparin containing tubes for naloxone PK prior to dosing and 2.5, 5, 10, 15, 20, 30, 45, 60, 120, 180, 240, 300, 360, 480, and 720 min after the start of study drug administration. Non-compartmental PK parameters including Cₘₐₓ, Tₘₐₓ, AUC to infinity (AUC_{0-∞}), AUC to last measurable concentration (AUC₀₋ₜ), t_{1/2}, λ_{z}, and apparent clearance (CL/F) were determined. Values of t_{1/2} were determined from the log-linear decline in plasma concentrations from 2 to 6 or 8 h.

Safety Evaluation: Heart rate, blood pressure, and respiration rate was recorded before naloxone dosing and at approximately 30, 60, 120, and 480 min after dosing. These vital signs and temperature were also measured at screening, clinic intake, one day after each dosing session and at follow-up. A 12-lead ECG was obtained prior to and approximately 60 and 480 min after each naloxone dose, as well as during screening, clinic intake, and follow-up. ECG and vital signs were taken within the 10-min period before the nominal time for blood collections. AEs were recorded from the start of study-drug administration until clinic discharge. AEs were recorded relative to each dosing session to attempt to establish a relationship between the AE and type of naloxone dose administered. An examination of the nasal passage was conducted at Day-1 to establish eligibility and at pre-dose, 5 min, 30 min, 60 min, 4 h, and 24 h post naloxone administration to evaluate evidence of irritation to the nasal mucosa. Clinical laboratory measurements were done prior to the first drug administration and on the day of clinic release.

Statistical Analysis of PK Parameters: Cₘₐₓ, Tₘₐₓ and AUC for 2 and 4 mg IN naloxone were compared with those for 0.4 mg IM naloxone. Within an ANOVA framework, comparisons of natural log (LN) transformed PK parameters (Cₘₐₓ and AUC) for IN versus IM naloxone treatments were performed. The 90% confidence interval (CI) for the ratio (IN/IM) of the least squares means of AUC and Cₘₐₓ parameters was constructed. These 90% CI were obtained by exponentiation of the 90% confidence intervals for the difference between the least squares means based upon a LN scale. In addition, dose adjusted values for AUCs and Cₘₐₓ based upon a 0.4 mg dose were calculated (Tables 4-7). The relative extent of absorption (relative bioavailability, Fᵣₑₗ) of intranasal (IN versus IM) was estimated from the dose-corrected AUCs.

Statistical Analysis of Adverse Events: AEs were coded using the most recent version of the Medical Dictionary for Regulatory Activities (MedDRA). Preferred terms and are grouped by system, organ, class (SOC) designation. AEs are presented as a listing including the start date, stop date, severity, relationship, outcome, and duration.

Pharmacokinetics Results: The mean dose delivered for the 2 mg IN naloxone dose was 1.71 mg (range 1.50 mg to 1.80 mg) and for the 4 mg IN naloxone dose it was 3.40 mg (range 2.93 mg to 3.65 mg). This was 84-85% of the target dose. The overall % coefficient of variation (% CV) for the delivered dose from all 42 devices was 6.9% (Table 9). Preparation time of the IN doses took less than one third of the time to prepare the IM injection (70 seconds for the IM injection and 20 seconds for the IN administration) (Table 8). The time to prepare the IM injection did not include loading the syringe. Since the one purpose of the study was to determine if peak naloxone plasma concentrations (Cₘₐₓ) and AUCs following IN 2 mg and IN 4 mg administrations were equivalent to, or greater than IM 0.4 mg dosing, AUCs and Cₘₐₓ values were compared without considering the dose difference among treatments. The Cₘₐₓ, AUC₀₋ₜ, and AUC_{0-∞}. for both the 2 mg IN and 4 mg IN doses were statistically significantly greater than those for the 0.4 mg IM dose (p<0.001). The geometric least square means for Cₘₐₓ were 2.18 ng/mL, 3.96 ng/mL, and 0.754 ng/mL for IN 2 mg, IN 4 mg and IM 0.4 mg, respectively. The geometric least square means for AUC_{0-∞} were 3.32 ng·h/mL, 5.47 ng·h/mL and 1.39 ng·h/mL for IN 2 mg, IN 4 mg and IM 0.4 mg respectively. The geometric least squares mean ratios for IN 2 mg/IM 0.4 mg were 290% for Cₘₐₓ and 239% for AUC_{0-∞}. The ratios for IN 4 mg/IM 0.4 mg were 525% for Cₘₐₓ and 394% for AUC_{0-∞}. There were no statistically significant differences between the routes and doses with respect to Tₘₐₓ, suggesting peak effects would occur at similar times for all treatments. However, the mean Tₘₐₓ values did trend lower for the IN route versus IM, and for 4 mg IN versus 2 mg IN. (See Table 2). In comparing the extent of systemic absorption of IN to IM dosing, the Fᵣₑₗ estimates were 55.7% and 46.3% for IN 2 mg and 4 mg, respectively. See Table 3.

Safety Results: No erythema, edema, erosion, or other sign was observed in the nasal cavity prior to or after any IN administration of naloxone at 2 and 4 mg to both nostrils. One subject experienced mild transient (over 3 min) pharyngeal pain coincident with the application of the 2 mg IN dose. This pain resolved spontaneously. Vital signs, ECG, and clinical laboratory parameters did not reveal any clinically noteworthy changes after naloxone administration. There was no evidence of QTcF prolongation.

TABLE 1
Order of Naloxone Doses and Route
of Administration for each Subject

| | Sub- | | | | | | | |
|---|---|---|---|---|---|---|---|---|
| | ject | Sequence | Dosing Session | | Dosing Session | | Dosing Session | |
| # | ID | # | #1 Day 1 | | #2 Day 5 | | #3 Day 9 | |
| 1 | 102 | 5 | 4 | mg IN | 2 | mg IN | 0.4 | mg IM |
| 2 | 107 | 6 | 0.4 | mg IM | 4 | mg IN | 2 | mg IN |
| 3 | 112 | 1 | 2 | mg IN | 4 | mg IN | 0.4 | mg IM |
| 4 | 117 | 3 | 0.4 | mg IM | 2 | mg IN | 4 | mg IN |
| 5 | 120 | 1 | 2 | mg IN | 4 | mg IN | 0.4 | mg IM |
| 6 | 123 | 2 | 4 | mg IN | 0.4 | mg IM | 2 | mg IN |
| 7 | 127 | 3 | 0.4 | mg IM | 2 | mg IN | 4 | mg IN |
| 8 | 128 | 5 | 4 | mg IN | 2 | mg IN | 0.4 | mg IM |
| 9 | 133 | 2 | 4 | mg IN | 0.4 | mg IM | 2 | mg IN |
| 10 | 113 | 4 | 2 | mg IN | 0.4 | mg IM | 4 | mg IN |
| 11 | 114 | 1 | 2 | mg IN | 4 | mg IN | 0.4 | mg IM |
| 12 | 119 | 6 | 0.4 | mg IM | 4 | mg IN | 2 | mg IN |
| 13 | 125 | 4 | 2 | mg IN | 0.4 | mg IM | 4 | mg IN |
| 14 | 135 | 5 | 4 | mg IN | 2 | mg IN | 0.4 | mg IM |

TABLE 2
Summary of Naloxone Pharmacokinetic Parameters Following
Naloxone as 0.4 mg Intramuscular (IM), 2 mg Intranasal
(IN), and 4 mg IN Administrations

| | 0.4 mg IM | | 2 mg IN | | 4 mg IN | |
|---|---|---|---|---|---|---|
| Parameter | Mean | % CV | Mean | % CV | Mean | % CV |
| Dose (mg) | 0.400 | — | 1.714 | 5.7 | 3.403 | 5.7 |
| Cₘₐₓ (ng/mL) | 0.765 | 27.6 | 2.32 | 41.2 | 4.55 | 63.7 |
| Tₘₐₓ (min) | 20.34 | 36.1 | 19.98 | 31.0 | 18.42 | 33.6 |
| AUC₀₋ₜ ng · | 1.38 | 19.9 | 3.41 | 29.5 | 5.63 | 27.6 |
| h/mL | | | | | | |
| AUC_{0-∞} (ng · | 1.42 | 19.2 | 3.44 | 29.3 | 5.68 | 27.6 |
| h/mL) | | | | | | |
| λ_{z} (1/h) | 0.593 | 16.6 | 0.588 | 0.572 | 8.0 | 10.2 |
| t_{1/2} (h) | 1.21 | 20.1 | 1.19 | 8.3 | 1.22 | 10.2 |

TABLE 3
Summary of Naloxone Pharmacokinetic Parameters Following Naloxone
as 0.4 mg Intramuscular (IM), 2 mg Intranasal (IN), and 4 mg
IN Administrations with Dose Normalized to 0.4 mg

| | 0.4 mg IM | | 2 mg IN | | 4 mg IN | |
|---|---|---|---|---|---|---|
| Parameter | Mean | % CV | Mean | % CV | Mean | % CV |
| AUC_{0-t/D} ng · h/mL | 1.38 | 19.9 | 0.796 | 28.7 | 0.667 | 29.4 |
| AUC_{0-∞/D} ng · h/mL | 1.42 | 19.2 | 28.5 | 0.674 | 0.804 | 29.3 |
| Fᵣₑₗ | | | 0.571 | 24.5 | 0.475 | 25.3 |

TABLE 4
Statistical Comparison of Geometric Least Squares Mean (GLSM)
of Pharmacokinetic Parameters for IN Naloxone at a Dose of 2
mg to IM Naloxone at a Dose of 0.4 mg with No Dose Adjustment

| | GLSM | GLSM | GLSM | | |
|---|---|---|---|---|---|
| | 2 mg | 0.4 mg | Ratio | 90% CI of | |
| Parameter | IN | IM | IM/IN % | Ratio | p-value |
| Cₘₐₓ (ng/mL) | 2.18 | 0.754 | 290 | 237-353 | <0.001 |
| Tₘₐₓ (h) 1.000 | 0.333 | 0.308 | — | — | — |
| AUC₀₋ₜ | 3.28 | 1.35 | 243 | 219-270 | <0.001 |
| (ng · h/mL) | | | | | |
| AUC_{0-∞} | 3.32 | 1.39 | 239 | 215-264 | <0.001 |
| (ng · h/mL) | | | | | |
| t_{1/2} (h) | 1.18 | 1.19 | 102 | 94.0-111 | 0.6507 |

TABLE 5
Statistical Comparison of Geometric Least Squares Mean (GLSM)
of Pharmacokinetic Parameters for IN Naloxone at a Dose of 4
mg to IM Naloxone at a Dose of 0.4 mg with No Dose Adjustment

| | GLSM | GLSM | GLSM | | |
|---|---|---|---|---|---|
| | 4 mg | 0.4 mg | Ratio | 90% CI of | |
| Parameter | IN | IM | IM/IN % | Ratio | p-value |
| Cₘₐₓ (ng/mL) | 3.96 | 0.754 | 525 | 431-640 | <0.001 |
| Tₘₐₓ (h) 1.000 | 0.292 | 0.308 | 0.418 | | |
| AUC₀₋ₜ (ng · h/mL) | 5.41 | 1.35 | 401 | 361-445 | <0.001 |
| AUC_{0-∞} (ng · h/mL) | 5.47 | 1.39 | 394 | 355-436 | <0.001 |
| t_{1/2} (h) | 1.22 | 1.19 | 102 | 94.0-111 | 0.651 |

TABLE 6
Statistical Comparison of Geometric Least Squares
Mean (GLSM) of Pharmacokinetic Parameters for IN
Naloxone at a Dose of 2 mg to IM Naloxone at a
Dose of 0.4 mg with Dose Adjustment to 0.4 mg

| | GLSM | GLSM | GLSM | | |
|---|---|---|---|---|---|
| | 2 mg | 0.4 mg | Ratio | 90% CI of | |
| Parameter | IN | IM | IM/IN % | Ratio | p-value |
| C_{max/D} (ng/mL) | 0.510 | 0.755 | 67.6 | 55.3-82.7 | 0.0028 |
| Tₘₐₓ (h) | 0.333 | 0.308 | — | — | 1.000 |
| AUC_{0-t/D} | 0.767 | 1.35 | 56.8 | 50.8-63.4 | <0.001 |
| (ng · h/mL) | | | | | |
| AUC_{0-∞/D} | 0.775 | 1.39 | 55.7 | 50.0-62.1 | <0.001 |
| (ng · h/mL) | | | | | |
| t_{1/2} (h) | 1.18 | 1.19 | 99.3 | 91.3-108 | 0.8963 |

TABLE 7
Statistical Comparison of Comparison of Geometric
Least Squares Mean (GLSM) Pharmacokinetic Parameters
for IN Naloxone at a Dose of 4 mg to IM Naloxone at
a Dose of 0.4 mg with Dose Adjustment to 0.4 mg

| | GLSM | GLSM | GLSM | | |
|---|---|---|---|---|---|
| | 4 mg | 0.4 mg | Ratio | 90% CI of | |
| Parameter | IN | IM | IM/IN % | Ratio | p-value |
| C_{max/D} (ng/mL) | 0.466 | 0.755 | 61.7 | 50.5-75.5 | <0.001 |
| Tₘₐₓ (h) | 0.292 | 0.308 | — | — | 0.418 |
| AUC_{0-t/D} | 0.637 | 1.35 | 47.2 | 42.2-52.7 | <0.001 |
| (ng · h/mL) | | | | | |
| AUC_{0-∞/D} | 0.644 | 1.39 | 46.3 | 41.5-51.6 | <0.001 |
| (ng · h/mL) | | | | | |
| t_{1/2} (h) | 1.22 | 1.19 | 102 | 94.0-111 | 0.651 |

TABLE 8
Time to Prepare the IM and IN Doses for Administration

| | Time (seconds) | | |
|---|---|---|---|
| | IM Dose | 2 mg IN Dose | 4 mg IN Dose |
| N | 14 | 14 | 14 |
| Mean | 70 | 19 | 23 |
| SD | 10 | 4 | 3 |
| Median | 73 | 19 | 23 |
| Minimum | 50 | 15 | 18 |
| Maximum | 82 | 30 | 28 |

TABLE 9
Estimated IN Dose Delivered (mg)

| | | | | | All |
|---|---|---|---|---|---|
| | 2 mg Dose | 4 mg Dose | | | Devices |
| | Total | First Device | Second Device | Total | Total |
| N | 14 | 14 | 14 | 14 | 42 |
| Mean | 1.697 | 1.682 | 1.687 | 3.369 | 1.689 |
| SD | 0.097 | 0.156 | 0.092 | 0.193 | 0.116 |
| % CV | 5.7 | 9.3 | 5.4 | 5.7 | 6.9 |
| Median | 1.708 | 1.711 | 1.704 | 3.410 | 1.710 |
| Minimum | 1.481 | 1.315 | 1.506 | 2.898 | 1.315 |
| Maximum | 1.838 | 1.824 | 1.803 | 3.616 | 1.838 |

### Example 2

### Pharmacokinetics and Safety of Intranasal Naloxone in Humans (Study 2)

A second study was undertaken to determine the pharmacokinetics (PK) and bioavailability of intranasally-delivered naloxone compared to intramuscularly-injected naloxone.

Objectives.

Specifically, the study had several objectives. The first was to determine the pharmacokinetics (i.e., the Cₘₐₓ, Tₘₐₓ, AUC_{0-inf} and AUC₀₋ₜ) of 4 intranasal doses—2 mg, 4 mg (2 nostrils), 4 mg (1 nostril), and 8 mg (2 nostrils)—of naloxone compared to a 0.4 mg dose of naloxone administrated IM and to identify an appropriate IN dose that could achieve systemic exposure comparable to an approved parenteral dose. The second was to determine the pharmacokinetics of two different concentrations (20 mg/mL and 40 mg/mL) of IN naloxone. The third was to determine the safety of IN naloxone, including adverse events, vital signs, and clinical laboratory changes, specifically with respect to nasal irritation (erythema, edema, and erosion).

Design.

The study was an inpatient open-label, randomized, 5-period, 5-treatment, 5-sequence, crossover study involving approximately 30 healthy volunteers, randomized to have at least 24 subjects who complete all study drug administrations and blood collections for PK assessments. Subjects were assigned to one of the 5 sequences and there were 6 subjects in each. Each subject received 5 naloxone treatments during the 5 dosing periods: a single 2 mg IN dose (one 0.1 mL spray of a 20 mg/mL solution in one nostril), a 4 mg IN dose (one 0.1 mL spray of a 20 mg/mil, solution in each nostril), a single 4 mg IN close (one 0.1 mL spray of a 40 mg/mL solution in one nostril), a single 8 mg IN dose (one 0.1 mL spray of a 40 mg/mL solution in each nostril), and a single 0.4 mg IM dose. Subjects stayed in an inpatient facility for 18 days to complete the entire study and were discharged on the next day after the last dose. Subjects returned for a final follow-up visit 3 to 5 days after discharge.

After obtaining informed consent, subjects were screened for eligibility to participate in the study including medical history, physical examination, clinical chemistry, coagulation markers, hematology, infectious disease serology, urinalysis, urine drug and alcohol toxicology screen, vital signs and ECG.

Inclusion criteria were: men or women 18 to 55 years of age, inclusive; written informed consent; BMI ranging from 18 to 30 kg/m2, inclusive; adequate venous access; no clinically significant concurrent medical conditions; agreement to use a reliable double-barrier method of birth control from the start of screening until one week after completing the study (oral contraceptives are prohibited); and agreement not to ingest alcohol, drinks containing xanthine >500 mg/day, or grapefruit/grapefruit juice, or participate in strenuous exercise 72 hours prior to admission through the last blood draw of the study.

Exclusion criteria were: any IN conditions including abnormal nasal anatomy, nasal symptoms (i.e., blocked and/or runny nose, nasal polyps, etc.), or having a product sprayed into the nasal cavity prior to drug administration; taking prescribed or over-the-counter medications, dietary supplements, herbal products, vitamins, or recent use of opioid analgesics for pain relief (within 14 days of last use of any of these products); positive urine drug test for alcohol, opioids, cocaine, amphetamine, methamphetamine, benzodiazepines, tetrahydrocannabinol (THC), barbiturates, or methadone at screening or admission; previous or current opioid, alcohol, or other drug dependence (excluding nicotine and caffeine), based on medical history; subject consumes greater than 20 cigarettes per day on average, in the month prior to screening, or would be unable to abstain from smoking (or use of any nicotine-containing substance) for at least one hour prior to and 2 hours after naloxone dosing; on standard 12-lead ECG, a QTcF interval >440 msec for males and >450 msec for females; significant acute or chronic medical disease in the judgment of the investigator; a likely need for concomitant treatment medication during the study; donated or received blood or underwent plasma or platelet apheresis within the 60 days prior to the day before study commencement; female who is pregnant, breast feeding, or plans to become pregnant during the study period or within one week after naloxone administration; positive test for hepatitis B surface antigen (HBsAg), hepatitis C virus antibody (HCVAb) or human immunodeficiency virus antibody (HIVAb) at screening; and current or recent (within 7 days prior to screening) upper respiratory tract infection.

Naloxone for IM injection manufactured by Hospira was obtained from a licensed distributor at a concentration of 0.4 mg/mL and was given IM at a dose of 0.4 mg in 1.0 mL with a 23-g needle as a single injection in the gluteus maximus muscle. Naloxone for IN administration was obtained from Lightlake Therapeutics, Inc., London, United Kingdom at two concentrations of 20 mg/mL and 40 mg/mL, and was given as doses of 2 mg (one 0.1 mL spray of the 20 mg/mL formulation in one nostril), 4 mg (two 0.1 mL sprays of the 20 mg/mL formulation in two nostrils), 4 mg (one 0.1 mL spray of the 40 mg/mL formulation in one nostril) and 8 mg (two 0.1 mL sprays of the 40 mg/mL formulation in two nostril). IN naloxone was administered using an Aptar single dose device with the subject in a fully supine position. Subjects were to be instructed to not breathe through the nose when the IN dose of naloxone was administered.

On the day after clinic admission, subjects were administered study drug in randomized order with a 4-day washout period between doses until all 5 treatments were administered. Blood was collected for naloxone PK prior to dosing and approximately 2.5, 5, 10, 15, 20, 30, 45, 60, 120, 180, 240, 300, 360, 480 and 720 minutes after the start of study drug administration, into sodium heparin containing tubes. On days of study drug administration, a 12-lead ECG was performed approximately 60 minutes prior to dosing and at approximately 60 and 480 minutes post-dose. Vital signs were measured pre-dose and approximately 30, 60, 120, and 480 minutes post-dose. On dosing days, the order of assessments were ECG, vital signs, then PK blood collection when scheduled at the same nominal times. The target time of the PK blood collection was considered the most important, and if the collection was more than ±1 minute from the scheduled time for the first 60 minutes of collections or more than ±5 minutes for the scheduled time points thereafter, this was considered a protocol deviation. ECG and vital signs were collected within the 10 minute period before the nominal time of blood collections. At screening, admission, discharge, and follow-up, ECG and vital signs were checked once per day. Vital signs were also checked once on the day after naloxone administration. Clinical laboratory measurements were repeated after the last PK blood draw prior to clinic discharge. Adverse events were assessed by spontaneous reports by subjects, by examination of the nasal mucosa, by measuring vital signs, ECG, and clinical laboratory parameters.

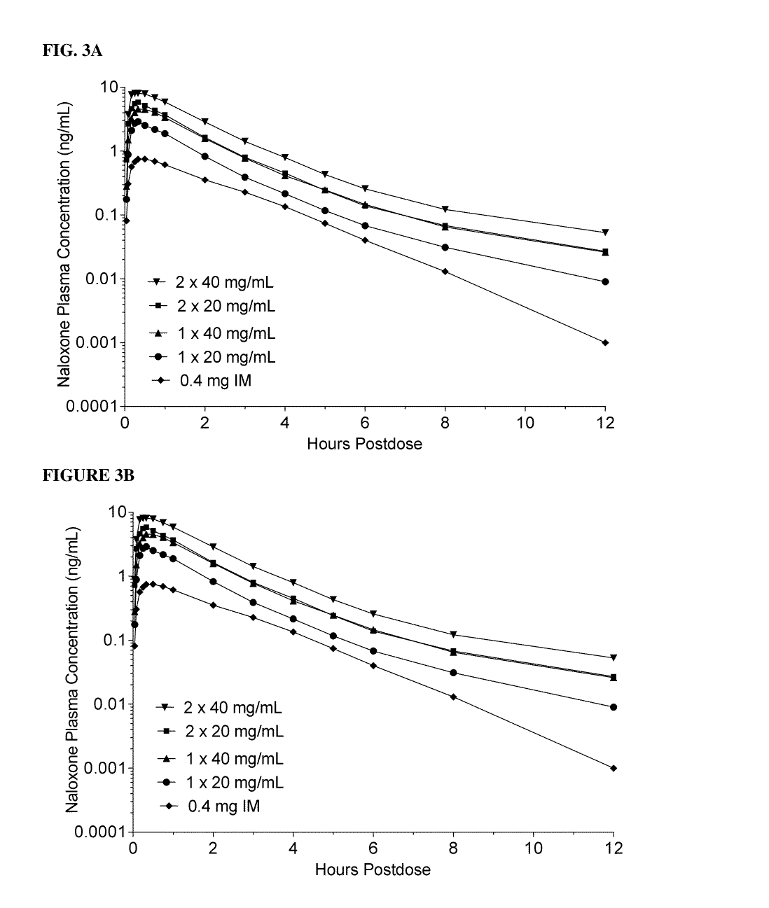

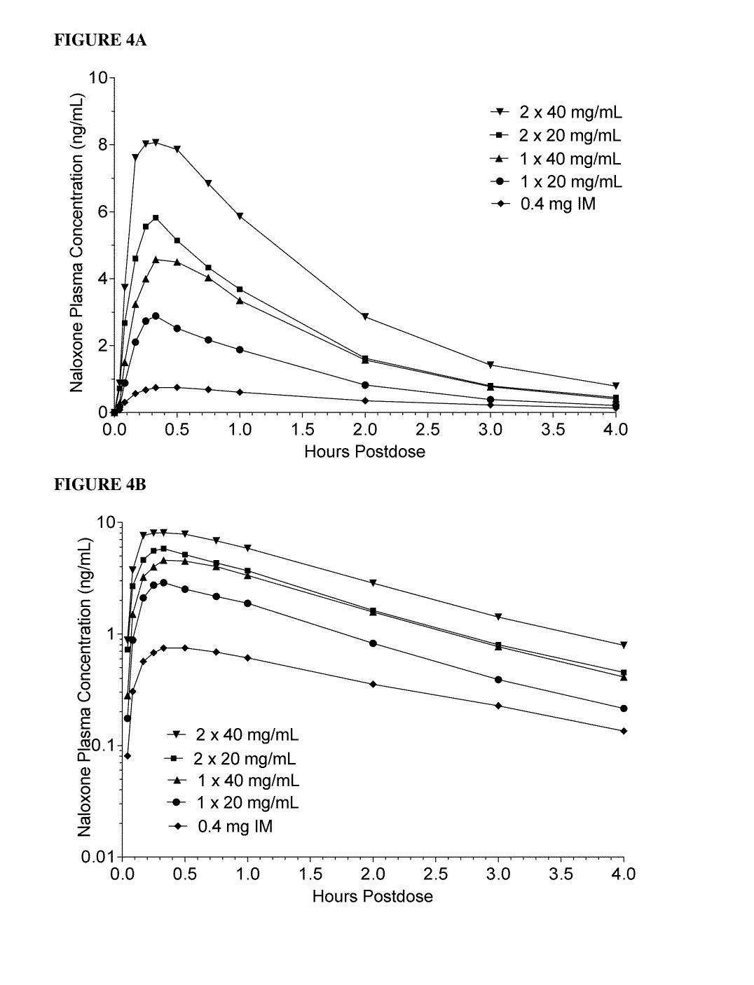

Results are shown below in Table 9, which sets forth the mean from 28 healthy subjects (and SD, in parentheses) plasma concentrations of naloxone following single intranasal administrations and an intramuscular injection, and in FIGS. 3 and 4.

TABLE 9

| | One Spray - 2 | | Two Sprays - 4 | | One Spray - 4 | | Two Sprays - 8 | | | |
|---|---|---|---|---|---|---|---|---|---|---|
| Time | mg | | mg | | mg | | mg | | | |
| (min) | 20 mg/mL IN | | 20 mg/mL IN | | 40 mg/mL IN | | 40 mg/mL IN | | 0.4 mg IM | |
| 0 | 0.000 | (0.000) | 0.000 | (0.000) | 0.000 | (0.000) | 0.000 | (0.000) | 0.000 | (0.000) |
| 2.5 | 0.175 | (0.219) | 0.725 | (0.856) | 0.280 | (0.423) | 0.880 | (1.21) | 0.081 | (0.135) |
| 5 | 0.882 | (0.758) | 2.68 | (2.65) | 1.50 | (1.76) | 3.73 | (4.02) | 0.305 | (0.336) |
| 10 | 2.11 | (1.33) | 4.60 | (2.59) | 3.24 | (2.21) | 7.61 | (5.28) | 0.566 | (0.318) |
| 15 | 2.74 | (1.07) | 5.56 | (2.20) | 4.00 | (2.24) | 8.02 | (3.60) | 0.678 | (0.312) |
| 20 | 2.89 | (1.14) | 5.82 | (1.74) | 4.57 | (2.30) | 8.06 | (2.56) | 0.747 | (0.271) |
| 30 | 2.52 | (0.810) | 5.15 | (1.70) | 4.50 | (1.93) | 7.89 | (1.95) | 0.750 | (0.190) |
| 45 | 2.17 | (0.636) | 4.33 | (1.16) | 4.03 | (1.57) | 6.84 | (1.69) | 0.689 | (0.171) |
| 60 | 1.88 | (0.574) | 3.69 | (0.887) | 3.35 | (1.17) | 5.86 | (1.40) | 0.610 | (0.143) |
| 120 | 0.823 | (0.335) | 1.63 | (0.626) | 1.57 | (0.773) | 2.86 | (0.927) | 0.354 | (0.107) |
| 180 | 0.390 | (0.146) | 0.800 | (0.253) | 0.771 | (0.412) | 1.42 | (0.487) | 0.227 | (0.082) |
| 240 | 0.215 | (0.100) | 0.452 | (0.225) | 0.412 | (0.215) | 0.791 | (0.275) | 0.135 | (0.058) |
| 300 | 0.117 | (0.051) | 0.243 | (0.123) | 0.246 | (0.143) | 0.431 | (0.166) | 0.074 | (0.047) |
| 360 | 0.068 | (0.030) | 0.139 | (0.067) | 0.146 | (0.081) | 0.257 | (0.104) | 0.040 | (0.022) |
| 480 | 0.031 | (0.014) | 0.068 | (0.033) | 0.065 | (0.038) | 0.122 | (0.052) | 0.013 | (0.015) |
| 720 | 0.009 | (0.009) | 0.027 | (0.013) | 0.026 | (0.019) | 0.053 | (0.025) | 0.001 | (0.003) |

For pharmacokinetic analysis, plasma was separated from whole blood and stored frozen at ≦−20° C. until assayed. Naloxone plasma concentrations was determined by liquid chromatography with tandem mass spectrometry. Conjugated naloxone plasma concentrations may also be determined. Non-compartmental PK parameters including Cₘₐₓ, Tₘₐₓ, AUC_{0-inf}, AUC₀₋ₜ, t_{1/2}, λ_{z}, and apparent clearance (CL/F) were determined. Pharmacokinetic parameters (Cₘₐₓ, Tₘₐₓ, and AUCs) for IN naloxone were compared with those for IM naloxone. Tₘₐₓ was from the time of administration (spraying into the nasal cavity or IM injection). Dose adjusted values for AUCs and Cₘₐₓ were then calculated, and the relative extent of intranasal absorption (IN versus IM) estimated from the dose-corrected AUCs. Within an ANOVA framework, comparisons of ln-transformed PK parameters (Cₘₐₓ and AUC) for intranasal versus IM naloxone treatments were performed. The 90% confidence interval for the ratio (IN/IM) of the geometric least squares means of AUC and Cₘₐₓ parameters were constructed for comparison of each treatment with IM naloxone. These 90% CIs were obtained by exponentiation of the 90% confidence intervals for the difference between the least squares means based upon an ln scale.

Results are shown below in Table 10, which sets forth the mean plasma PK parameters from 28 healthy subjects (and % CV, in parentheses) of naloxone following single intranasal administrations and an intramuscular injection, and in Table 11, which sets forth the same PK parameters split between the 12 female and 16 male healthy subjects.

TABLE 10

| | One Spray - | Two Sprays - | One Spray - | Two Sprays - | |
|---|---|---|---|---|---|
| Parameter | 2 mg 20 | 4 mg 20 | 4 mg 40 | 8 mg 40 | |
| (units) | mg/mL IN | mg/mL IN | mg/mL IN | mg/mL IN | 0.4 mg IM |
| Cₘₐₓ (ng/ml) | 3.11 (36.3) | 6.63 (34.2) | 5.34 (44.1) | 10.3 (38.8) | 0.906 (31.5) |
| Cₘₐₓ per mg | 1.56 (36.3) | 1.66 (34.2) | 1.34 (44.1) | 1.29 (38.8) | 2.26 (31.5) |
| (ng/mL) | | | | | |
| Tₘₐₓ (h)^{a} | 0.33 (0.25, | 0.33 (0.08, | 0.50 (0.17, | 0.33 (0.17, | 0.42 (0.08, |
| (median, range) | 1.00) | 0.50) | 1.00) | 1.00) | 2.00) |
| AUCₜ | 4.81 (30.3) | 9.82 (27.3) | 8.78 (37.4) | 15.9 (23.6) | 1.79 (23.5) |
| (ng · mL/h) | | | | | |
| AUC_{inf} | 4.86 (30.1) | 9.91 (27.1) | 8.87 (37.2) | 16.1 (23.3) | 1.83 (23.0) |
| (ng · mL/h) | | | | | |
| AUC_{inf} per mg | 2.43 (30.1) | 2.48 (27.1) | 2.22 (37.2) | 2.01 (23.3) | 4.57 (23.0) |
| (ng · mL/h) | | | | | |
| Lambda z | 0.3685 | 0.2973 | 0.3182 | 0.3217 | 0.5534 |
| (hr⁻¹)^{b} | | | | | |
| Half-life (h)^{b} | 1.70 | 2.09 | 2.00 | 1.91 | 1.19 |
| AUC % | 1.09 (41.9) | 1.01 (53.9) | 1.06 (52.5) | 1.04 (78.1) | 2.32 (54.1) |
| Extrapolate | | | | | |
| CL/F (L/h) | 441 (24.5) | 426 (22.3) | 502 (31.2) | 521 (21.7) | 230 (22.4) |
| Relative BA | 53.8 (22.2) | 55.3 (22.2) | 49.2 (30.6) | 45.3 (25.1) | 100 |
| (%) vs. IM | | | | | |

TABLE 11

| Parameter | One 20 mg/mL IN | | Two 20 mg/mL IN | | One 40 mg/mL IN | | Two 40 mg/mL IN | | 0.4 mg IM | |
|---|---|---|---|---|---|---|---|---|---|---|
| (units) | Female | Male | Female | Male | Female | Male | Female | Male | Female | Male |
| Cₘₐₓ | 2.79 | 3.35 | 6.62 | 6.64 | 5.12 | 5.51 | 9.52 | 10.9 | 1.06 | 0.792 |
| (ng/ml) | | | | | | | | | | |
| Cₘₐₓ per | 1.39 | 1.68 | 1.66 | 1.66 | 1.28 | 1.38 | 1.19 | 1.36 | 2.64 | 1.98 |
| mg | | | | | | | | | | |
| (ng/mL) | | | | | | | | | | |
| Tₘₐₓ (h)^{a} | 0.33 | 0.33 | 0.33 | 0.25 | 0.50 | 0.50 | 0.29 | 0.42 | 0.33 | 0.50 |
| AUCₜ | 4.73 | 4.87 | 9.81 | 9.82 | 7.98 | 9.38 | 14.8 | 16.8 | 1.83 | 1.75 |
| (ng · mL/h) | | | | | | | | | | |
| AUC_{inf} | 4.78 | 4.93 | 9.91 | 9.92 | 8.06 | 9.48 | 15.0 | 16.9 | 1.88 | 1.79 |
| (ng · mL/h) | | | | | | | | | | |
| AUC_{inf} per | 2.39 | 2.46 | 2.48 | 2.48 | 2.01 | 2.37 | 1.87 | 2.12 | 4.69 | 4.47 |
| mg | | | | | | | | | | |
| (ng · mL/h) | | | | | | | | | | |
| Lambda z | 0.3978 | 0.3492 | 0.2796 | 0.3122 | 0.2946 | 0.3386 | 0.2994 | 0.3407 | 0.6140 | 0.5152 |
| (hr⁻¹)^{b} | | | | | | | | | | |
| Half-life | 1.58 | 1.80 | 2.18 | 2.03 | 2.12 | 1.93 | 1.90 | 1.91 | 1.08 | 1.28 |
| (h) ^{b} | | | | | | | | | | |
| AUC % | 0.971 | 1.19 | 0.986 | 1.02 | 0.970 | 1.12 | 1.12 | 0.992 | 2.31 | 2.32 |
| Extrapolate | | | | | | | | | | |
| CL/F (L/h) | 449 | 434 | 419 | 431 | 555 | 462 | 558 | 494 | 222 | 236 |

In the tables above, the notation a indicates median (range) is disclosed, and the notation b indicates harmonic mean is disclosed.

Additional exploratory analyses could include:
1) 90% CI for dose corrected AUC and Cₘₐₓ between the 20 mg/mL formulation treatment and 40 mg/mL formulation for both a single administration and two dose administration (once in each nostril) for dose linearity purpose;
2) 90% CI adjusted for dose for geometric ratios of one 0.1 mL spray (in one nostril) vs. a two 0.1 mL sprays (one spray in each nostril) from an 20 mg/mL formulation; and
3) 90% CI adjusted for dose for geometric ratios of one 0.1 mL spray (in one nostril) vs. a two 0.1 mL sprays (one spray in each nostril) from an 40 mg/mL formulation;

AEs were coded using the most recent version of the Medical Dictionary for Regulatory Activities (MedDRA) preferred terms and grouped by system, organ, class (SOC) designation. Separate summaries will be provided for the 5 study periods: after the administration of each dose of study drug up until the time of the next dose of study drug or clinic discharge. Listings of each individual AE including start date, stop date, severity, relationship, outcome, and duration were provided. Results are given below in Tables 12 and 13. Table 12 shows the events related to nasal irritation—erythema, edema, other, and total—observed in the nasally-treated group. Nasal irritation did not appear to be positively related to the dose of naloxone given.

TABLE 12

| Treatment | Erythema | Edema | Other | Total |
|---|---|---|---|---|
| 2 mg (20 mg/mL, one spray) | 4 | 2 | 1 | 7 |
| 4 mg (20 mg/mL, two sprays) | 1 | 0 | 0 | 1 |
| 4 mg (40 mg/mL, one spray) | 1 | 2 | 0 | 3 |
| 8 mg (40 mg/mL, two sprays) | 0 | 1 | 0 | 1 |

Table 1e shows additional events related to administration either nasally or intramuscularly. Overall, few adverse events were reported.

TABLE 13

| 0.4 mg Intramuscular Dose | |
|---|---|
| Dizziness | 1 |
| Headache | 1 |
| Nausea | 1 |
| 2 mg (20 mg/mL, one spray) | |
| Nasal Pain | 1 |
| 8 mg (40 mg/mL, two sprays) | |
| Headache | 1 |

Additionally, vital signs, ECG, and clinical laboratory parameters did not reveal any clinically noteworthy changes after naloxone administration. There was no evidence of QTcF prolongation.

### Example 3

### Naloxone Nasal Spray Formulations and Stability

Naloxone has been formulated as a disposable Luer-Jet Luer-lock pre-filled syringe and nasal atomizer kit product, comprising 1 mg/ml naloxone hydrochloride as an active agent, 8.35 mg/ml NaCl as an isotonicity agent, HCl q.s. to target pH, and purified water q.s. to 2.0 ml. Benzalkonium chloride may be added as a preservative and supports the stability of a multi-dose product. Such syringes, while functional, can be ungainly to use by untrained personnel, and deliver a large volume of solution.

Examples of a 10 mg/ml formulation are given below in Table 14.

TABLE 14

| Ingredient | Quantity per unit | Function |
|---|---|---|
| Naloxone hydrochloride | 10 mg/ml | Active ingredient |
| Sodium chloride | 7.4 mg/ml | Isotonicity agent |
| Hydrochloric acid | q.s. to target pH | Acidifying agent |
| Benzalkonium chloride | 0.1 mg/ml | Preservative |
| Purified water | q.s. | Solvent |

Literature data has indicated that naloxone is sensitive to environmental factors, such as air, light and colours in certain vials, which may induce a risk for degradation. Consequently disodium edetate was added to the above formulation.

Pharmaceutical compositions comprising naloxone hydrochloride (10 mg/mL) were stored at 25° C. and 60% relative humidity in upright clear glass vials (200 μL) stoppered with a black plunger. Vials were either nude (Batch 1), or mounted in the Pfeiffer BiDose device (Batch 2). In addition to naloxone hydrochloride, the pharmaceutical compositions further comprised water, benzalkonium chloride, and disodium edetate. The vials were assayed at 0, 3, 6, 9, and 12 months for naloxone content. It is evident from the results of the study, reported as a percentage of the label claim in Table 15 below, that these pharmaceutical compositions are storage-stable for at least 9-12 months at 25° C. and 60% relative humidity.

TABLE 15

| | Time (months) | | | | |
|---|---|---|---|---|---|
| Batch | 0 | 3 | 6 | 9 | 12 |
| 1 | 99.3 | 100.1 | 100.8 | 101.2 | 97.9 |
| 2 | 99.5 | 102.8 | 99.4 | 98.6 | ND |

Examples of 20 mg/ml and a 40 mg/ml formulation are given below in Table 16, along with an example of permitted variation as part of the total formulation.

TABLE 16

| | 20 mg/ml | | 40 mg/ml | | |
|---|---|---|---|---|---|
| | | Quantity | | Quantity | |
| | | per unit | | per unit | |
| Concentration | Quantity | dose | Quantity | dose | Product |
| Component | per ml | (100 μl) | per ml | (100 μl) | Variation |
| Naloxone HCl | 22.0 mg | 2.2 mg | 44.0 mg | 4.4 mg | 90.0-110.0 |
| dihydrate | (20.0 mg) | (2.0 mg) | (40.0 mg) | (40.0 mg) | |
| (corresponding | | | | | |
| to naloxone HCl) | | | | | |
| Benzalkonium | 0.1 mg | 0.01 mg | 0.1 mg | 0.01 mg | 90.0-110.0 |
| chloride | | | | | |
| Disodium edetate | 2.0 mg | 0.2 mg | 2.0 mg | 0.2 mg | 80.0-120.0 |
| Sodium chloride | 7.4 mg | 0.74 mg | 7.4 mg | 0.74 mg | |
| Hydrochloric | Adjust to | Adjust to | Adjust to | Adjust to | pH 3.5-5.5 |
| acid, dilute | pH 4.5 | pH 4.5 | pH 4.5 | pH 4.5 | |
| Purified water | q.s. ad 1.0 | q.s. ad 100 | q.s. ad | q.s. ad 100 | |
| | ml | μl | 1.0 ml | μl | |

The naloxone hydrochloride nasal spray above is an aqueous solution which may be presented in a Type I glass vial closed with a chlorobutyl rubber plunger which in turn is mounted into a unit-dose nasal spray device (such as an Aptar UDS liquid UnitDose device). The solution should be a clear and colorless or slightly yellow liquid. In certain embodiments, the device is a non-pressurized dispenser delivering a spray containing a metered dose of the active ingredient. In certain embodiments, each delivered dose contains 100 μl.

Pharmaceutical compositions comprising naloxone hydrochloride (20 or 40 mg/mL) were tested for stability in room temperature/light conditions, room temperature/dark conditions and in 25° C./60% RH (protected from light). It was tested for pH, purity, and impurities at an initial time point, 2 months and 10 months. Results are given in Table 17.

TABLE 17

| | Test | | | Assay (% | |
|---|---|---|---|---|---|
| Storage | interval | | | of label | Impurities |
| condition | (months) | Appearance | pH | claim) | (area %) |
| | Initial | Clear, | 4.5 | 101 | Not |
| | | colourless | | | detected |
| | | solution | | | |
| 25° C./ | 2 | Not | 45 | Not | Not |
| 60% RH | | analyzed | | analyzed | analyzed |
| | 10 | Clear, | 4.5 | 95 | 0.2 |
| | | colourless | | | |
| | | solution | | | |
| Room | 10 | Clear, yellow | 4.4 | 92 | 1.3 |
| temper- | | solution | | | |
| ature/light | | | | | |
| Room | 10 | Clear, | 4.5 | 97 | 0.3 |
| temper- | | colourless | | | |
| ature/dark | | solution | | | |

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1-194. (canceled)

195. A drug product comprising a combination of a therapeutically effective amount of an opioid agonist and a therapeutically effective amount of naloxone hydrochloride or a hydrate thereof,
wherein said naloxone hydrochloride or hydrate thereof is contained in a pre-primed device adapted for nasal delivery of a pharmaceutical composition to a patient by actuation of said device into a nostril of said patient, and
wherein the pre-primed device comprises at least one reservoir, each reservoir containing a pharmaceutical composition which is an aqueous solution of about 100 μL comprising:
between about 2 mg and about 12 mg of the naloxone hydrochloride or a hydrate thereof;
between about 0.2 mg and about 1.2 mg of an isotonicity agent;
between about 0.005 mg and about 0.015 mg of a preservative;
between about 0.01 mg and about 0.05 mg of a stabilizing agent; and
an amount of acid sufficient to achieve a pH or 3.5-5.5.

196. The drug product as recited in claim 195, wherein the pre-primed device is a single-dose, pre-primed device,
wherein the pharmaceutical composition is present in one reservoir, and
wherein the therapeutically effective amount of the opioid antagonist is delivered by one actuation of the drug delivery device into one nostril of the patient.

197. The drug product as recited in claim 196, wherein the pre-primed device adapted comprises about 2 mg, about 4 mg, or about 8 mg of the naloxone hydrochloride or a hydrate thereof.

198. The drug product as recited in claim 197, wherein:
the isotonicity agent is NaCl;
the preservative is benzalkonium chloride;
the stabilizing agent is disodium edetate; and
the acid is hydrochloric acid.

199. The drug product as recited in claim 198, wherein the aqueous solution comprises:
about 4.4 mg naloxone hydrochloride dihydrate;
about 0.74 mg NaCl;
about 0.01 mg benzalkonium chloride;
about 0.2 mg disodium edetate; and
an amount of hydrochloric acid sufficient to achieve a pH or 3.5-5.5.

200. The drug product as recited in claim 199, wherein the pre-primed device is a single-use, pre-primed device.

201. The drug product as recited in claim 196, wherein the agonist and the therapeutically effective amount of naloxone hydrochloride or a hydrate thereof contained in the pre-primed device are co-packaged.

202. The drug product as recited in claim 201, wherein the package additionally comprises instructions to administer the naloxone hydrochloride or a hydrate thereof to the patient while the patient is in the supine position.

203. The drug product as recited in claim 196, wherein the opioid agonist is selected from the group consisting of: alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof.

204. The drug product as recited in claim 203, wherein the opioid agonist is selected from the group consisting of hydrocodone and oxycodone.

205. The drug product of claim 195, wherein the pre-primed device is a pre-primed, bi-dose device,
wherein a first volume of said pharmaceutical composition is present in a first reservoir, and a second volume of said pharmaceutical composition is present in a second reservoir, and
wherein said therapeutically effective amount of said opioid antagonist is delivered essentially by a first actuation of said drug delivery device from said first reservoir into a nostril of said patient and a second actuation of said drug delivery device from said second reservoir into a nostril of said patient.

206. The drug product as recited in claim 205, wherein each reservoir of the pre-primed device comprises about 2 mg, about 4 mg, or about 8 mg of the naloxone hydrochloride or a hydrate thereof.

207. The drug product as recited in claim 206, wherein:
the isotonicity agent is NaCl;
the preservative is benzalkonium chloride;
the stabilizing agent is disodium edetate; and
the acid is hydrochloric acid.

208. The drug product as recited in claim 207, wherein the aqueous solution comprises:
about 2.2 mg or about 4.4 mg naloxone hydrochloride dihydrate;
about 0.74 mg NaCl; about 0.01 mg benzalkonium chloride;
about 0.2 mg disodium edetate; and
an amount of hydrochloric acid sufficient to achieve a pH or 3.5-5.5.

209. The drug product as recited in claim 208, wherein each reservoir comprises about 2.2 mg of the naloxone hydrochloride dihydrate.

210. The drug product as recited in claim 208, wherein each reservoir comprises about 4.4 mg of the naloxone hydrochloride dihydrate.

211. The drug product as recited in claim 208, wherein the pre-primed device is a single-use, pre-primed device adapted for nasal delivery of a pharmaceutical composition.

212. The drug product as recited in claim 205, wherein the agonist and the therapeutically effective amount of naloxone hydrochloride or a hydrate thereof contained in the pre-primed device are co-packaged.

213. The drug product as recited in claim 212, wherein the package additionally comprises instructions to administer the naloxone hydrochloride or a hydrate thereof to the patient while the patient is in the supine position.

214. The drug product as recited in claim 205, wherein the opioid agonist is selected from the group consisting of: alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof.

215. The drug product as recited in claim 214, wherein the opioid agonist is selected from the group consisting of hydrocodone and oxycodone.

216. A method of lowering opioid overdose risk in an individual, comprising:
providing to the individual a combination of a therapeutically effective amount of an opioid agonist and a therapeutically effective amount of naloxone hydrochloride or a hydrate thereof,
wherein said naloxone hydrochloride or hydrate thereof is contained in a pre-primed device adapted for nasal delivery of a pharmaceutical composition to a patient by actuation of said device into a nostril of said patient, and
wherein the pre-primed device comprises at least one reservoir, each reservoir containing a pharmaceutical composition which is an aqueous solution of about 100 μL comprising:
between about 2 mg and about 12 mg of the naloxone hydrochloride or a hydrate thereof;
between about 0.2 mg and about 1.2 mg of an isotonicity agent;
between about 0.005 mg and about 0.015 mg of a preservative;
between about 0.01 mg and about 0.05 mg of a stabilizing agent; and
an amount of acid sufficient to achieve a pH or 3.5-5.5.

217. The method as recited in claim 216, wherein the pre-primed device is a single-dose, pre-primed device,
wherein the pharmaceutical composition is present in one reservoir, and
wherein the therapeutically effective amount of the opioid antagonist is delivered by one actuation of the drug delivery device into one nostril of the patient.

218. The method as recited in claim 217, wherein the pre-primed device comprises about 2 mg, about 4 mg, or about 8 mg of the naloxone hydrochloride or a hydrate thereof.

219. The method as recited in claim 218, wherein:
the isotonicity agent is NaCl;
the preservative is benzalkonium chloride;
the stabilizing agent is disodium edetate; and
the acid is hydrochloric acid.

220. The method as recited in claim 219, wherein the aqueous solution comprises:
about 4.4 mg naloxone hydrochloride dihydrate;
about 0.74 mg NaCl;
about 0.01 mg benzalkonium chloride;
about 0.2 mg disodium edetate; and
an amount of hydrochloric acid sufficient to achieve a pH or 3.5-5.5.

221. The method as recited in claim 220, wherein the pre-primed device is a single-use, pre-primed device.

222. The method as recited in claim 217, further comprising:
providing instruction to a user to administer the therapeutically effective amount of naloxone hydrochloride or a hydrate thereof contained in the pre-primed device to a patient in a supine position.

223. The method as recited in claim 217, wherein the opioid agonist is selected from the group consisting of: alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof.

224. The method as recited in claim 223, wherein the opioid agonist is selected from the group consisting of hydrocodone and oxycodone.

225. The method as recited in claim 216, wherein the pre-primed device is a pre-primed, bi-dose device, wherein a first volume of said pharmaceutical composition is present in a first reservoir, and a second volume of said pharmaceutical composition is present in a second reservoir, and wherein said therapeutically effective amount of said opioid antagonist is delivered essentially by a first actuation of said drug delivery device from said first reservoir into a nostril of said patient and a second actuation of said drug delivery device from said second reservoir into a nostril of said patient.

226. The method as recited in claim 225, wherein each reservoir of the pre-primed device comprises about 2 mg, about 4 mg, or about 8 mg of the naloxone hydrochloride or a hydrate thereof.

227. The method as recited in claim 226, wherein each reservoir comprises about 2 mg of the naloxone hydrochloride or a hydrate thereof.

228. The method as recited in claim 227, wherein each reservoir comprises about 4 mg of the naloxone hydrochloride or a hydrate thereof.

229. The method as recited in claim 228, wherein:
the isotonicity agent is NaCl;
the preservative is benzalkonium chloride;
the stabilizing agent is disodium edetate; and
the acid is hydrochloric acid.

230. The method as recited in claim 229, wherein each reservoir comprises:
about 2.2 mg or about 4.4 mg naloxone hydrochloride dihydrate;
about 0.74 mg NaCl; about 0.01 mg benzalkonium chloride; and
about 0.2 mg disodium edetate.

231. The method as recited in claim 230, wherein the pre-primed device is a single-use, pre-primed device.

232. The method as recited in claim 225, further comprising the step of providing instruction to a user to administer the therapeutically effective amount of naloxone hydrochloride or a hydrate thereof contained in the pre-primed device to a patient in a supine position.

233. The method as recited in claim 225, wherein the opioid agonist is selected from the group consisting of: alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, and tramadol, and pharmaceutically acceptable salts thereof.

234. The method as recited in claim 233, wherein the opioid agonist is selected from the group consisting of hydrocodone and oxycodone.

235. The method as recited in claim 216, wherein the individual is an individual at risk for opioid overdose.

236. The method as recited in claim 216, wherein the individual is a patient with a take-home opioid prescription.
